SEQUENCE LISTING (4 sequences)

SEQ ID NO: 1 - protein, 6 aa, Artificial Sequence
  Synthesized
STVIIE

SEQ ID NO: 2 - protein, 6 aa, Artificial Sequence
  Synthesized
QVQIIE

SEQ ID NO: 3 - protein, 6 aa, Artificial Sequence
  Synthesized
ISFLIF

SEQ ID NO: 4 - protein, 7 aa, Artificial Sequence
  Synthesized
GNNQQNY

(19) United States
(12) Patent Application Publication
(10) Pub. No.: US 2023/0184756 A1
(43) Pub. Date: Jun. 15, 2023

(54) BIOMATERIALS

(71) Applicant: INSTITUTO DE MEDICINA MOLECULAR JOÃO LOBO ANTUNES, Lisbon (PT)

(72) Inventors: Ivo Martins, Lisbon (PT); Gabriela Guerra, Santo Tirso (PT); Patricia Carvalho, Abitureiras (PT)

(21) Appl. No.: 17/760,441

(22) PCT Filed: Feb. 10, 2021
(86) PCT No.: PCT/EP2021/053235

§ 371 Date: Aug. 9, 2022

(30) Foreign Application Priority Data
Feb. 10, 2020 (PT) 116112
Feb. 20, 2020 (GB) 2002343.8

(51) Int. Cl. G01N 33/543; G01N 33/569; G01N 33/535

(52) U.S. Cl. CPC G01N 33/54386; G01N 33/56916; G01N 33/535

(57) ABSTRACT

This invention relates to a functionalised biomaterial comprising aggregated self-assembling peptides, for example amyloidogenic peptides, such as STVIIE, QVQIIE, ISFLIF and/or GNNQQNY, wherein at least a proportion of the self-assembling peptides are functionalised with a biological agent or a chemical agent. The self-assembling peptides may be connected to reporter molecules and recognition elements, such as antibodies. These biomaterials allow for signal amplification for example in assays for detecting analytes. Biomaterials, assays and kits are provided.

### PRIORITY

This application is a 371 and claims benefit of European Application No. EP2021/053235, filed Feb. 10, 2021, which claims benefit of Portugal Application No. 116112, filed Feb. 10, 2020, and United Kingdom Application No. 2002343.8, filed Feb. 20, 2020, which applications are incorporated herein by reference in their entirety.

### INCORPORATION BY REFERENCE OF SEQUENCE LISTING

A Sequence Listing is provided herewith as a Sequence Listing text, MEWE-111_SEQLIST_2023-02-28 created on Feb. 28, 2023 and having a size of 1,078 bytes. The contents of the Sequence Listing text are incorporated herein by reference in their entirety.

### FIELD

This invention relates to materials made from aggregated peptides, which are useful for sampling and testing analytes. In particular, the invention relates to biomaterials comprising aggregated peptides, devices comprising those materials, and methods of using the materials and devices for sampling, analysing or detecting analytes.

### BACKGROUND

Amyloid fibrils are originated via a process designated amyloidogenesis, by which peptide or protein monomers spontaneously self-assemble into higher order aggregates namely oligomers, protofibrils and, finally, the mature amyloid fibrils. The first studies of amyloidogenesis were performed mainly in the context of the so-called amyloid diseases, such as Alzheimer's and Parkinson's syndrome, among others¹⁻¹⁰. Due to the association with these pathologies, it was initially thought that mature amyloid fibrils are toxic by themselves and just the result of a random misfolding process. However, several studies showed that toxicity is more often associated with precursor aggregates, i.e., oligomers and protofibrils, than with the mature amyloid fibrils and that amyloidogenesis is actually a highly ordered process^{1-3,11-13}. Furthermore, subsequent investigations demonstrated that amyloid species are involved in several physiological processes, performing functional roles such as extracellular matrix materials of fungi and bacteria¹⁴⁻¹⁶, as protective envelops of insects and fish eggs^{17,18}, and other roles, as reviewed elsewhere¹⁹⁻²³. Remarkably, amyloid-like nanofibrils seem to be the functional structure of stored peptide hormones^{24,25}, being also involved in melanin formation in humans^{26,27}.

This fresh understanding of amyloid fibrils physiological roles sparked a renewed interest in their study, not only in the framework of misfolding studies but also as possible novel biomaterials. This potential has been previously overlooked but, in fact, amyloid fibrils possess key characteristics that make them appropriate biomaterials for nanotechnology applications^{19,28}. A key requirement is stability, allowing for chemical reactions to occur in the immediate vicinity of the biomaterials without affecting them¹⁹. Another major requirement is the ability of the biomaterial to be chemically modified for a specific function, without affecting chemical and mechanical stability¹⁹. It is also desirable in some applications for a biomaterial that self-assembles in a well-established and ordered manner to produce diverse topographies^{19-21,28}. Amyloid fibrils possess these exact features and are hence becoming attractive tools in nanotechnology, with potential applications across various fields, including the biomedical sciences, as novel ordered nanomaterials^{19-21,28-31}.

One of the most important characteristics of amyloid fibrils is their particular highly ordered structure^{1,13,32-35}. Fibrillar aggregates derived from different amyloidogenic peptide/protein sequences share common features, being composed of β-sheet structure stabilized by hydrogen-bonds between adjacent β-strands that run perpendicular along the fibril axis^{1,3,13,19,36}. The distances between β-strands are mostly determined by the size of the amino acid side chains¹⁹. The mature amyloid fibril is then formed by the assembly of the β-strands protofilaments^{1,19,36-40.} A wealth of data on these processes is now available. Due to this, conceiving new amyloid species for special purposes has become viable, by taking advantage of design algorithms based on empirical and theoretical rules governing amylodogenesis^{8,28,41-46}.

Given the above, amyloid fibrils are excellent candidate biomaterials. Short amyloidogenic peptide sequences have structural compatibility, nanoscale dimensions, organized assembly into well-defined nanostructures, low cost and easy of production (of constituent monomers), allowing various technological developments, including for bio-sensing uses ^{19-21,28,39,47,48, 60-65}. For this purpose, it is important to make use of amyloidogenic peptides that form stable amyloid fibrils in physiological conditions of pH and temperature (in which most biologically relevant interactions and processes occur) and that can be easily derivatized with specific chemical moieties (to add them new functions, when needed). Moreover, having different amyloid topologies, to suit different applications, would be desirable.

### SUMMARY OF INVENTION

The invention is based on the surprising development of new biomaterials from simple peptides. The peptides can assembled into a fibril or gel biomaterial. Typically, the peptides are able to self-assemble into the biomaterial under appropriate conditions. The peptides can be functionalised, for example with a biological or chemical molecule, without preventing this assembly into the higher-order structure of the biomaterial. Although the monomeric peptides themselves are typically soluble in aqueous and/or physiological conditions, the resulting aggregated biomaterial is surprisingly able to maintain its structure over hours, days or weeks. The provision of an easily assembled stable biomaterial is therefore particularly advantageous.

The biomaterial can be labelled with multiple reporter molecules, so that a single binding event between the functionalised biomaterial and a target analyte provides multiple reporter signals. This can advantageously be used to amplify the signal from a single binding event. Furthermore, the ability to provide a fully-formed biomaterial, or to allow self-assembly of the components to form the biomaterial where it is needed, provides a highly adaptable material and, for example, allows for signal amplification in situ with minimal sample processing.

A first aspect of the invention provides a functionalised biomaterial comprising aggregated self-assembling peptides, wherein at least a proportion of the self-assembling peptides are functionalised with a biological agent or a chemical agent. The biomaterial may, in certain embodiments, be a fibril or gel. Typically, the self-assembling peptides are able to self-assemble under physiological conditions, preferably the self-assembling peptides are able to self-assemble spontaneously under physiological conditions (for example 20-40° C., atmospheric pressure of 1, pH 6-8). The assembled biomaterial is typically non-toxic.

The self-assembling peptides may be amyloidogenic peptides.

In certain embodiments, the biomaterial is composed of a peptide that comprises, consists of, or consists essentially of: STVIIE, QVQIIE, ISFLIF and/or GNNQQNY. In some embodiments, 1, 2, 3 conservative substitutions may be made to the peptide provided that the self-assembling properties are retained. Conservative substitutions are known in the art and are typically accepted as being a substitution within the same general class of amino acid residue, as summarised in the table below:

| Class | Amino acids |
|---|---|
| Aliphatic | Glycine, Alanine, Valine, Leucine, |
| | Isoleucine |
| Hydroxyl or sulphur/selenium | Serine, Cysteine, Selenocysteine, |
| containing | Threonine, Methionine |
| Cyclic | Proline |
| Aromatic | Phenylalanine, Tyrosine, Tryptophan |
| Basic | Histidine, Lysine, Arginine |
| Acidic/amides | Aspartate, Glutamate, Asparagine, |
| | Glutamine |

1, 2 or 3 insertions or deletions to the STVIIE, QVQIIE, ISFLIF and/or GNNQQNY peptides may also be made, provided that their self-assembling properties are retained.

The peptide may be part of a larger peptide, provided that the self-assembling properties are retained. For example, at least 1, 2, 3, or more additional amino acid residues may be present at one end or at both ends of the STVIIE, QVQIIE, ISFLIF and/or GNNQQNY. Typically, a longer peptide comprising the STVIIE, QVQIIE, ISFLIF and/or GNNQQNY peptide contains a maximum of 20 amino acid residues, for example 15 or fewer amino acid residues, typically 10 or fewer amino acid residues.

The peptides that form the biomaterial may be provided homogenously, or a heterogeneous mix of different peptides. All of the peptides may be peptides of the invention, for example STVIIE, QVQIIE, ISFLIF and/or GNNQQNY peptides, or other peptides may optionally be included. In some embodiments a majority of STVIIE peptides are used, or only STVIIE peptides are used, some or all of which may be functionalised.

Typically, the majority (e.g. >50%, >60%, >70%, >80%, >90% or more) of peptides are functionalised, for example all or substantially all of the peptides are functionalised, although the biomaterial still functions in assays even when only a minority of the peptides (<50%) are functionalised.

In exemplary embodiments, a fibril incorporates 60%-90% unfunctionalised peptide (e.g. STVIIE) with 10% to 40% functionalised peptide (weight/weight), for example 60%-80% unfunctionalised peptide with 20% to 40% functionalised peptide (weight/weight), about 65%-75% unfunctionalised peptide with 25% to 35% functionalised peptide (weight/weight), or about 70% unfunctionalised functionalised with about 30% functionalised peptide (weight/weight). Other amyloidogenic sequences, mixed in similar or different ratios, of free peptide and biotinylated versions, are provided to give a similar result to those demonstrated herein. In certain embodiments, the peptides form amyloid fibrils with a periodic width of between 100 and 150 nm, a ΔX of between 150 nm and 200 nm, a ΔY of between 2 nm and 5 nm and a height of between 10 nm and 15 nm. In certain embodiments, the amyloid fibrils have a periodic width of 118±16 nm, a ΔX of 170±7 nm, a ΔY of 3.2±0.4 nm and a height of 13.5±0.9 nm.

The functionalised biomaterial is typically stable under physiological conditions. In some embodiments the biomaterial forms within six hours, and optionally is then allowed to develop further for another 24 hours to up to 2 weeks before being stable for use. A stable biomaterial retains its structure over the period in which it is needed, typically over a period of hours, days, weeks or months. This may be 1 week or more, 2 weeks or more, 3 weeks or more, 4 weeks or more, 6 weeks or more, or 8 weeks or more, for example 15 weeks or more. The physiological conditions are usually physiological pH and/or physiological temperature. Physiological pH is typically between 6 and 8, or between 6.5 and 7.5, or is about 7, or is about 7.4. Physiological temperature is between room temperature (e.g. around 20° C.) and human body temperature (about 3TC), for example 25° C. In some embodiments, the peptides of the invention are able to self-assemble at 20° C. in liquid water, or in general aqueous solutions with pH and salt concentrations at physiological levels at temperatures close to room temperature, when added in sufficient quantities and concentration, to form the aggregated biomaterial.

The biomaterial is functionalised. The term “functionalised” is to be given its usual meaning in the art, and relates to the inclusion of an additional functional molecule into the biomaterial. The functional molecule may be a chemical or biological agent. In some embodiments the chemical agent may be a vitamin, enzyme cofactor, reaction substrate, or catalyst. In some embodiments the biological agent may be a protein, nucleic acid or carbohydrate. In some embodiments, the biomaterial is functionalised by having biotin attached to at least a proportion of its component peptides. Biotin is well-known (IUPAC name 5-[(3aS,4S,6aR)-2-oxohexahydro-1H-thieno[3,4-d]imidazol-4-yl]pentanoic acid). Biotin is therefore an example of a suitable functionalising agent. The terms functionalising agent and functionalising element are used interchangeably herein. Other typical functionalising agents are proteins, for example a receptor, a ligand, or an antibody or antigen-binding fragment (e.g. Fab fragment) of an antibody, or a nucleic acid such as DNA or RNA. The functional molecule typically retains its required function when it is part of the biomaterial of the invention. In certain embodiments, the functional molecule in the biomaterial retains at least 50% of its function in free solution, for example at least 75% or at least 90%. In certain embodiments, the functional molecule in the biomaterial has substantially the same level of function as it has in free solution under equivalent conditions. Function may be determined by an appropriate assay depending on the functional molecule. For an enzyme or catalyst, the activity may be measured in enzyme units, katals (or nano- or micro-katals), or KM. For an antibody or other binding molecule, the function may be measured as the binding affinity, for example the dissociation constant as determined by surface plasmon resonance (for example the well-known Biacore assay) or another suitable assay.

The functionalising agent may be attached to a self-aggregating peptide of the invention using a linker. The linker may be rigid or flexible. The linker may be a heterobifunctional or a homobifunctional chemical linker.

The linker may be a glucoside linker, of which a suitable example is a TRIS-derived triglucoside (Tdts). In other embodiments, the linker may be a polyethylene glycol-based linker, for example PEG9 or PEG13.

In some embodiments, the functionalising agent may be attached directly to the peptide of the invention, for example by a covalent or non-covalent bond.

The functionalising agent may be attached (directly, or using a linker) anywhere on the peptide. In some embodiments, the functionalising agent is attached to the N-terminal half or the C-terminal half of the peptide. In some embodiments, the functionalising agent is attached to one of the 3 residues at either the N-terminus or the C-terminus of the peptide. In some embodiments, the functionalising agent is attached to the N-terminal (i.e. first) residue. In other embodiments, the functionalising agent is attached to the C-terminal (i.e. last) residue.

Suitable functionalising agents or molecules may include biotin.

At least one additional element may be connected to the functionalised biomaterial. Typically, a first additional element is referred to herein as a “bridge element”. This bridge element is optionally attached to the functionalising agent and, in this arrangement, the peptide is typically bound (e.g. covalently) to the functionalising agent which is in turn bound (e.g. non-covalently) to the bridge element. The bridge element may be a protein that binds specifically to the functionalising agent, for example it may be an antibody that binds specifically to an epitope on the functionalising agent (or it may comprise an epitope that binds specifically to an antibody functionalising agent). In some embodiments, the functionalising molecule and bridge element are a receptor and its ligand. The receptor and ligand can be in either orientation, such that the functionalising molecule is the receptor and the bridge element is the ligand, or such that the functionalising molecule is the ligand and the bridge element is the receptor. An example of a suitable bridge element is streptavidin, which can bind to a biotin functionalising agent.

The bridge element can be in solution or be immobilised to a surface, for example the surface of an assay chamber.

A reporter molecule can be bound (covalently or non-covalently) to at least one bridge element. A reporter molecule is typically detectable, and may be an optical reporter (such as a fluorescent or other optically-detectable label) or a chemical reporter, such as a catalyst or enzyme, that when present results in a detectable change in the system. An example of an enzyme reporter is horseradish peroxidase (HRP). As shown in the examples, HRP treated with substrate can be imaged via chemiluminescence.

In certain embodiments, a bridge element comprises multiple reporter molecules. In some embodiments, multiple reporter molecules are attached to each of at least 1, 2, 3, 4, 5 or more bridge elements. Multiple reporter molecules can be attached to multiple bridge elements, or attached to at least 50% of the bridge elements, or to all or substantially all of the bridge elements.

In certain embodiments, a bridge element comprises a single reporter molecule. In some embodiments, the biomaterial comprises at least 1, 2, 3, 4, 5 or more singly-labelled bridge elements. Multiple bridge elements can be labelled with a reporter molecule, for example at least 50%, at least 60%, at least 70% or at least 80% of the bridge elements, or in some embodiments all or substantially all of the bridge elements are labelled with a reporter molecule. Having multiple labelled bridge elements allows for amplification of the signal that is provided by the biomaterial.

In certain embodiments, the functionalising molecule or bridge element is specifically recognisable by a recognition element. The recognition element can be a protein, typically an antibody or receptor. The recognition element may also be a nucleic acid or any other molecule capable of selectively binding a target. In addition to recognising the functionalising molecule or bridge element, the recognition element is typically also able to bind specifically to an analyte of interest. Therefore, the recognition element typically has at least two binding sites, a first binding site for binding the functionalising molecule or the bridge element, and a second binding site or region for binding to an analyte. The recognition element may in some embodiments comprise or consist of a fusion protein of two antibodies, a fusion protein of an antibody and another binding protein, a fusion protein of at least two different proteins or protein domains, an antibody conjugate (such as an ADC or equivalent), or a bispecific or multispecific antibody. In some embodiments, the recognition element may be a streptavidin labelled antibody. If the target is for example a cell that expresses an Fc Receptor (e.g. a Natural Killer cell), then the recognition element could be an antibody with CDRs that bind specifically to the functionalising molecule or bridge element, and the Fc region will then engage the target cell.

When at least one bridge element comprises multiple reporter molecules, or when multiple bridge elements are labelled with a reporter molecule, recognition of the bridge element or the functionalising molecule by the recognition element (e.g. antibody) leads to multiple reporter molecules being captured for each single recognition event (e.g. antibody-ligand interaction), resulting in increased signal detection and amplification. In one embodiment set out in the Examples, the functionalising molecule is biotin, the bridge element is streptavidin, the reporter molecule is an enzyme (optionally HRP), and the recognition element is a streptavidin-labelled antibody. Other arrangements comprising 1, 2, 3 or all 4 of the functionalising molecule, bridge element, reporter molecule and recognition element can be prepared as required by the application of the technology.

According to a second aspect of the invention, there is provided the use of a biomaterial according to the first aspect, in a biological or chemical assay, for example an assay for the detection of an analyte.

A third aspect of the invention provides an assay to detect an analyte, wherein the assay comprises contacting the analyte with the biomaterial of the invention. The analyte may be contacted with a pre-formed biomaterial according to the first aspect, or the analyte may be contacted with one or more components of the biomaterial and the aggregation and assembly permitted to occur in situ. An embodiment wherein the separate components of the biomaterial are added to an analyte mixture is depicted in FIG. 10.

The assay may in certain embodiments be a diagnostic assay, a biosensing assay, an immunoassay, an immunodiagnostic assay, a dot blot or an ELISA. The biomaterial of the first aspect or its component parts may conveniently be provided as part of a biosensor or analytical apparatus, suitable for use in this assay, and optionally adapted specifically for use in the assay. The analyte that is detected or analysed may in some embodiments be a biological molecule such as a protein, carbohydrate or polynucleotide, a metabolite, a biomarker, a cell, a human cell, an animal cell, a plant cell, a microorganism, a bacteria, or a virus. In some embodiments, the analyte is from a biological sample, a fluid sample or a tissue sample. In certain embodiments, the assays of the second and third aspects may be to detect a biomarker, a diseased cell, or a pathogen. The pathogen may be a microorganism such as a bacteria, fungus, protozoa or worm. The pathogen may typically be detected in a sample from a patient suspected of carrying the pathogen, for example in a bodily fluid or tissue sample from the patient. The patient is typically human. Typical bacteria for detection may be gram negative or gram positive bacteria. The bacteria may be cocci such as Staphylococci, Streptococci (e.g. S. pneumonia) or Neisseriae (e.g. N. gonorrhoeae or N. meningitidis), gram positive bacilli such as Corynebacteria, Bacillus Anthracis, Listeria monocytogenes, gram negative bacilli such as Salmonella spp., Shigella, Campylobacter, Vibrio, Yersinia pestis, Pseudomas spp., Brucella, Haempohilus, Legionella or Bortedella. Other bacteria that can be detected include Mycobacteria such as M. tuberculosis, M. leprae or M. avium, Rickettsia, or Chlamydia.

In some embodiments, the pathogen may be a virus. Typical viruses that can be detected include: DNA viruses such as adenovirus, herpesvirus, poxvirus, parvovirus, papilloma virus or hepatitis, for example hepatitis B; or RNA viruses such as influenza, coronaviruses, paramyxovirus, picornavirus (e.g. polio, coxsackie, hepatitis A, rhinovirus), togaviruses (e.g. rubella), flaviviruses (e.g. causing yellow fever, dengue fever), rhabdoviruses (e.g. rabies), ebolavirus, or retroviruses such as HIV.

Fungi that can be detected include Candida albicans, Aspergillus or Pneumocystis.

Protozoa that can be detected include Leishmania, Plasmodium, Trypanosoma, Toxoplasma gondii or Crytosporidium.

In certain embodiments, the pathogen to be detected is a rhinovirus, coronavirus, influenza virus, adenovirus, or respiratory syncytial virus. These viruses can often cause symptoms known as the “common cold”.

In an exemplary embodiment, the pathogen to be detected is a coronavirus, more typically a human coronavirus such as the 2019-nCOV.

The biomaterial can be adapted to detect an analyte of interest. In certain embodiments, the biomaterial comprises an antigen-binding protein (e.g. an antibody or antigen-binding antibody fragment) that binds specifically to the analyte, thereby allowing for its detection from a mixture. When the analyte is a pathogen, the antibody or antigen-binding antibody fragment part of the biomaterial will typically bind to a component on the surface of the pathogen, that is typically characteristic for that pathogen, for example a surface protein or carbohydrate. When the analyte is an influenza virus, the biomaterial may typically comprise an antibody or fragment that specifically binds to neuraminidase or hemagluttinin. When the analyte is a coronavirus, the biomaterial may typically comprise an antibody or fragment that specifically binds to the spike protein or hemagluttinin-esterase dimer. When the analyte is a bacteria, for example Pneumococcus, the biomaterial may typically comprise an antibody or fragment that specifically binds to one or more cell capsule sugars. The analyte-specific component of the biomaterial (typically an antibody or antibody fragment) may in certain embodiments be incorporated as the functionalising agent that is attached to the self-assembling peptide. In some other embodiments, the analyte-specific component of the biomaterial (typically an antibody or antibody fragment) forms part of the bridge element.

Typically, the analyte-specific component of the biomaterial (typically an antibody or antibody fragment) forms part of the recognition element. In some of the examples below, the bridge element comprises streptavidin (to bind the biotin functionalising agent) and the recognition element comprises a analyte-binding antibody that also binds to the functionalising agent (by means of a streptavidin label on the analyte-recognising antibody). Typically, the recognition element recognises the analyte and binds to the biomaterial, thereby linking the biomaterial to the analyte.

In a fourth aspect, the invention provides an amyloidogenic peptide comprising, consisting or consisting essentially of the peptides STVIIE, QVQIIE, ISFLIF and/or GNNQQNY. In certain embodiments, the peptide at 1 mg/ml is soluble in liquid water at 20° C. In further embodiments, the amyloidogenic peptide is as described above for the first aspect, or as described elsewhere herein.

A fifth aspect of the invention provides a method of preparing a biomaterial, comprising providing amyloidogenic peptides according to the fourth aspect in conditions suitable for them to self-assemble, and allowing the peptides to self-assemble to form the biomaterial. In further embodiments, the biomaterial that is produced according to the fifth aspect is as described above for the first aspect, or as described elsewhere herein.

A sixth aspect of the invention provides a kit comprising one or more amyloidogenic peptides according to the fourth aspect and instructions for their self-assembly into a biomaterial. The kit may optionally further comprise:
i) instructions for performing the method of the fifth aspect; and/or
ii) instructions for performing the assay of the third aspect; and/or
iii) at least one bridge element, optionally labelled with at least one reporter molecule; and/or
iv) at least one recognition element that binds to the biomaterial, typically by binding to the bridge element or by binding to a peptide or a functionalising agent attached to the peptide;
   optionally wherein the recognition element specifically binds to an analyte such as a pathogen.

A seventh aspect of the invention provides a biosensor comprising a functionalised biomaterial according to the first aspect, or an amyloidogenic peptide according to fourth aspect. The biosensor may be used in the assay of the third aspect or provided as part of a kit according to the sixth aspect.

Other aspects and embodiments of the invention are described in more detail below.

### BRIEF DESCRIPTION OF THE FIGURES

FIG. 1 shows a generic description of an amyloid based signal amplification method. (a) Candidate amyloidogenic peptide monomers are tested, to determine those that form amyloid fibrils in physiologic conditions of pH, temperature and ionic strength (at which most physiological protein-ligands occur). Good candidate peptides are (b) functionalized with bioactive molecules bridge elements, via different linkers, and tested regarding their ability to (c) form stable fibrils, that are bound to functional bioactive molecules, connected via functional bridge elements (and adequate linker). Those fibrils that are functional and bioactive are then tested regarding the ability to hold several reporter molecules, both when (d) free in solution and when (e) immobilized. The approach has different applications, namely biomolecules detection and signal amplification, aiming at single-molecule detection based on multiple reporter molecules per fibril.

FIG. 2 shows GNNQQNY, QVQIIE, ISFLIF and STVIIE peptides amyloid morphology. Atomic force microscopy allows a nanoscale analysis of the amyloid fibrils formed at physiologic pH conditions. For all the peptides the images were acquired in different incubation times: 0 h of incubation (first column), 24 h of incubation (second column) and 2 weeks of incubation (last column). Overall peptides QVQIIE and GNNQQNY present structures that are not consistent with the desired amyloid-like fibril structures required for the signal amplification approach proposed. STVIIE and ISFLIF show amyloid fibril morphology. STVIIE are the most organized and form fibrils earlier and were thus selected.

FIG. 3 shows TVIIE morphology, dimensions, secondary structure, toxicity and binding to amyloid dyes. STVIIE peptide is highly structured forming well-defined fibrils with (a-c) typical amyloid morphology and (d-e) dimensions, as required and shown by atomic force microscopy (AFM). The scale bars of the AFM images in (a) and (b) correspond to 5 μm and 500 nm, respectively. AFM data treated with JPK analysis software v. 4.2.61 (JPK Instruments AG, Berlin, Germany). Regarding secondary structure content, (f) STVIIE amyloid fibrils are rich in β-sheet architecture, which (g) quickly stabilizes in less than 3 hours incubation, as desired and shown by Fourier transform infra-red (FTIR) spectroscopy. FTIR spectra were normalized and baseline corrected with OPUS Bruker data analysis software (Bruker Corporation, Germany). Importantly, mature fibrils (h) are not cytotoxic and bind amyloid dyes such as (i) Thioflavin T and (j) Congo Red, in a (k) concentration-dependent manner, for both dyes. Experiments conducted in triplicate.

FIG. 4 shows a selection of biotinylated amyloid fibril linkers. STVIIE biotinylated in its N-terminus via either a Tdts or a PEG9 linker incubated for 2 weeks in physiological conditions form (a-b) amyloid fibrils (as judged by AFM). Such fibrils and the biotin-conjugated versions are not toxic in H4 cells (c-d), that were treated with STVIIE species for 6 and 24 hours, as determined via LDH release viability assay (the data is the mean±SD of three independent measurements, after which ordinary one-way ANOVA followed by Tukey's multiple comparison test revealed no significant differences). Only amyloid fibrils of PEG9-biotin-STVIIE bind (e) Congo Red in a (f) concentration-dependent manner. PEG9-biotin-STVIIE was thus selected for the purposes stated. Experiments conducted in triplicate.

FIG. 5 shows a biotinylated STVIIE characterization and functional activity. PEG9-biotin-STVIIE fibrils (a) bind ThT and (b) display a FTIR spectra rich in β-sheet structure, similar to non-biotinylated STVIIE fibrils (see FIG. 4). Free streptavidin binds biotinylated STVIIE (via PEG9) amyloid fibrils as judged by (a-e) intrinsic tryptophan fluorescence studies. (c) Fluorescence intensity spectra of 0.8 μM streptavidin solution treated with buffer, free biotin, non-biotinylated free amyloid peptide and biotinylated amyloidogenic peptide raw spectra. Concentrations of peptide and/or biotin range from 0 to 13.6 μM (C0 to C11). The drop on fluorescence signal (decreased quantum yield), accompanied by peak maximum shifting, indicates streptavidin binding to biotin. (d) Fluorescence intensity normalized to the maximum peak of the spectra. (e) Differential intensity spectra between streptavidin and free biotin curves (left), and free non-biotinylated and biotinylated peptide (right). (f) Sum of the total area (negative and positive) from the biotin differential spectra (blue) and biotinylated peptide differential spectra (green), showing a typical saturation binding profile. (g) Peak maximum shift with the addition of free biotin (orange) or biotinylated peptide (red). The addition of biotinylated peptide to streptavidin produces a blue shift of the fluorescence emission maximum (from 340 to 330 nm) similar to the observed when free biotin is added. Overall, streptavidin fluorescence emission spectra in the presence of free biotin and of biotinylated peptide reveal similar profiles, as desirable. (h-i) Measuring biotin-PEG9-STVIIE fibrils binding to surface-immobilized streptavidin, via FTIR spectroscopy. Biotin-PEG9-STVIIE fibrils specifically bind (h) immobilized streptavidin, with the interaction being specific and consistent with (g) the presence of amyloid beta-sheet rich structure, as expectable (given the fibrils secondary structure). Experiments conducted in triplicate.

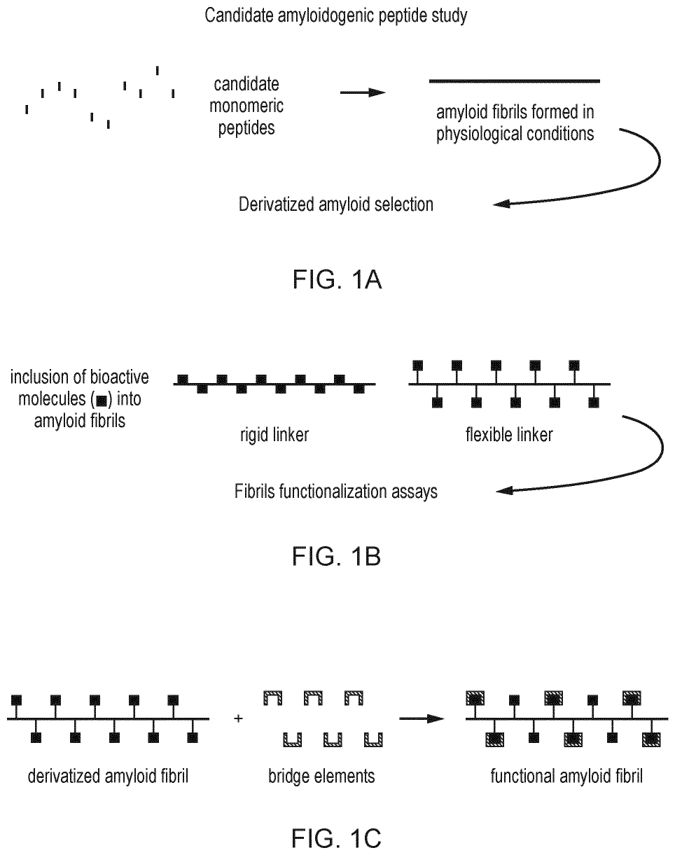

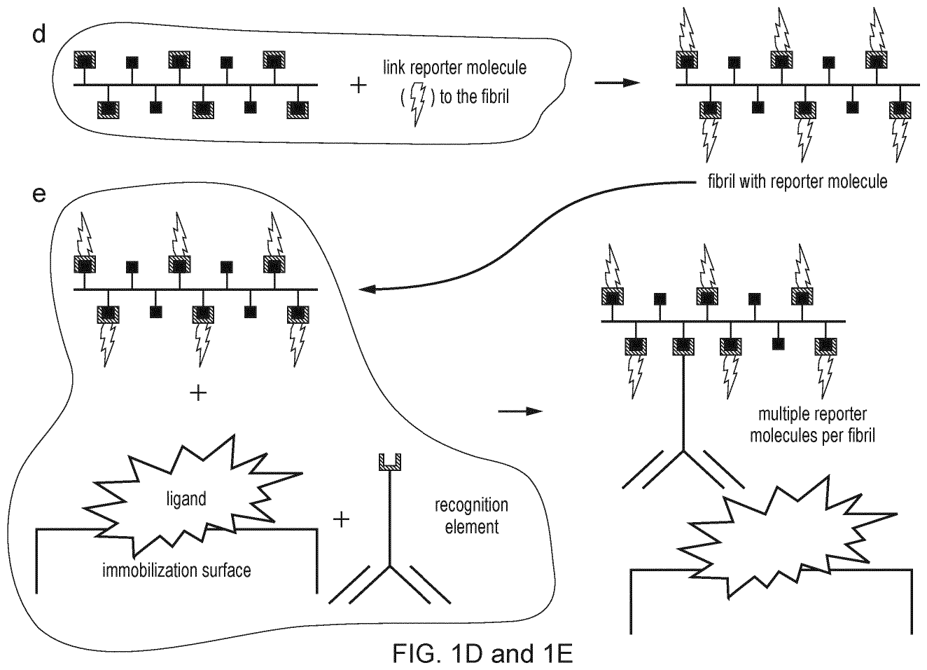

FIG. 1 shows detection of immobilized fibrils and immobilized proteins (IgG antibodies) via dot blot. Experimental (a) schematics and (b) results of detection of immobilized biotin-PEG9-STVIIE amyloid fibrils via immunochemistry dot blot chemiluminescence assays. The approach clearly detects (b) 8 ng and even 0.4 ng (faint signal) of immobilized fibrils. Experimental schematics (c-d) and (e-g) results of dot blot assays showing detection of immobilized ligands (antibodies). Briefly, using streptavidin labelled anti-IgG antibodies, plus PEG9 biotinylated STVIIE amyloid fibrils and then enzyme reporter molecules (horseradish peroxidise, HRP) derivatized with streptavidin (S-HRP), different primary antibodies, immobilized on a surface, are detectable by various secondary anti-IgG antibodies, raised against the IgG of each particular species. Briefly, it is shown: that (e) derivatized goat anti-IgG antibodies detect mouse, rabbit, and rat immobilized antibodies; that (f) derivatized rabbit anti-IgG antibodies detect mouse and rat immobilized antibodies; and, finally, that derivatized donkey anti-IgG antibodies detect mouse, rabbit, and rat immobilized antibodies. This demonstrates the approach general applicability, as a total of eight combinations of commonly used primary and secondary antibodies are detected by using the functionalized amyloid fibrils. Experiments conducted in triplicate.

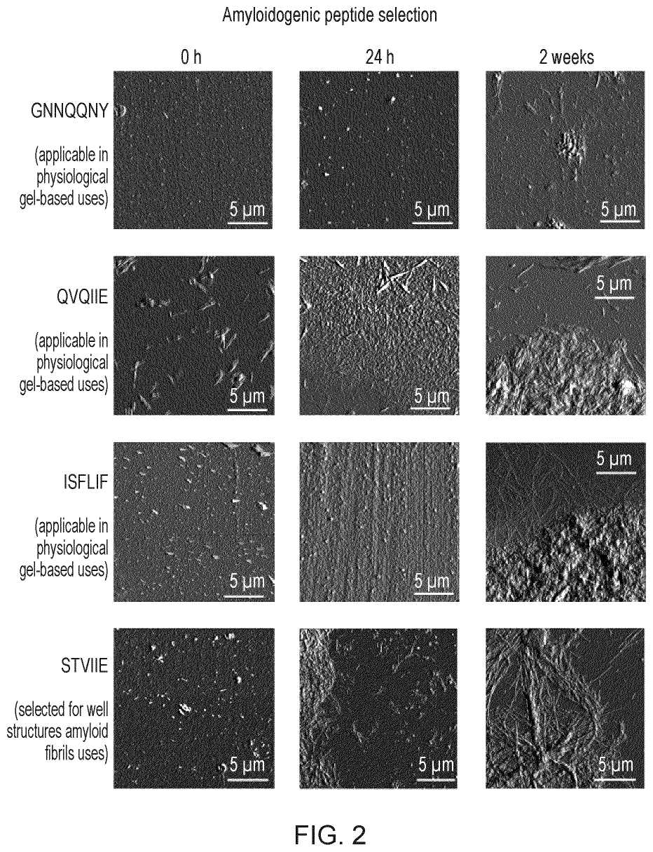

FIG. 2 shows detection of immobilized fibrils and of immobilized protein (GFAP) via dot blot. GFAP immobilized on a membrane surface is identified by a specific (a) streptavidin-derivatized anti-GFAP antibody and, then, biotin labelled amyloid fibrils are used for signal amplification of a single detection event, lowering the thresholds of detection, in a direct (with a single antibody) immunochemistry dot blot assay. The experiment also functions in (b) an indirect immunohistochemistry dot blot assay, using streptavidin-derivatized secondary anti-IgG antibody combined with an anti-GFAP antibody, in which case a higher signal amplification is produced, further lowering detection thresholds. Experiments conducted in triplicate, with biotin-PEG9-STVIIE fibrils added at 1 μg/mL Then, using a direct blot assay, in (c) liver and brain tissue samples from mice, detection of GFAP is achieved in (d) 10 and (e) 50 ng of total tissue sample (actual amount of GFAP is lower). GFAP is present at much higher levels in the brain, as expected. There is a concentration-dependent signal, as desirable, as observed when (d) 200 ng/mL (dilution 1:5000) and (e) 66 ng/mL (dilution 1:15000) of antibody are used. This supports the approach applicability in biopsy-like tissue samples.

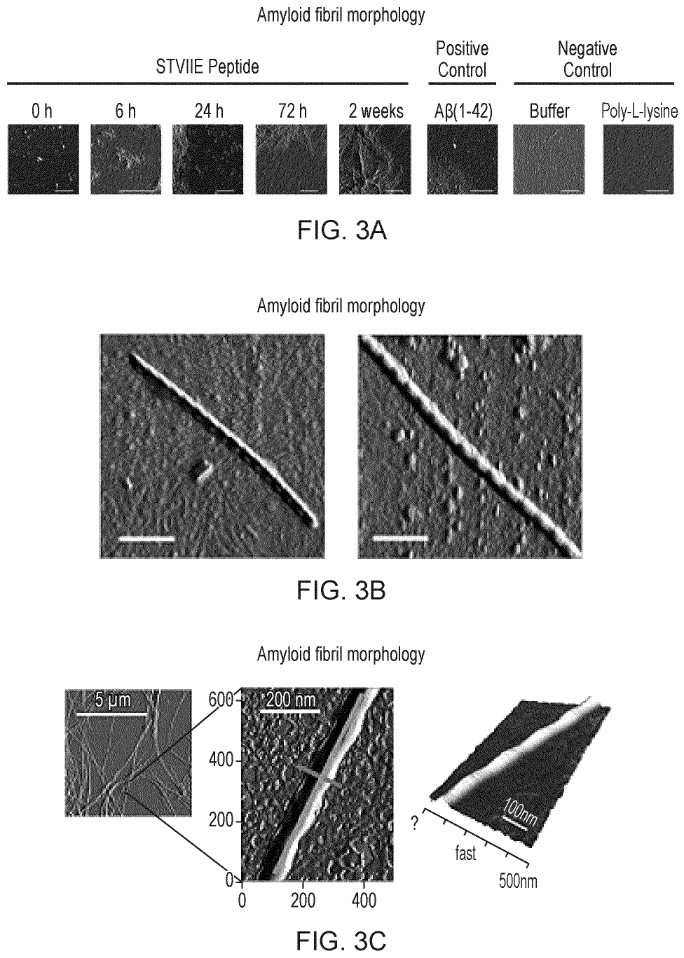

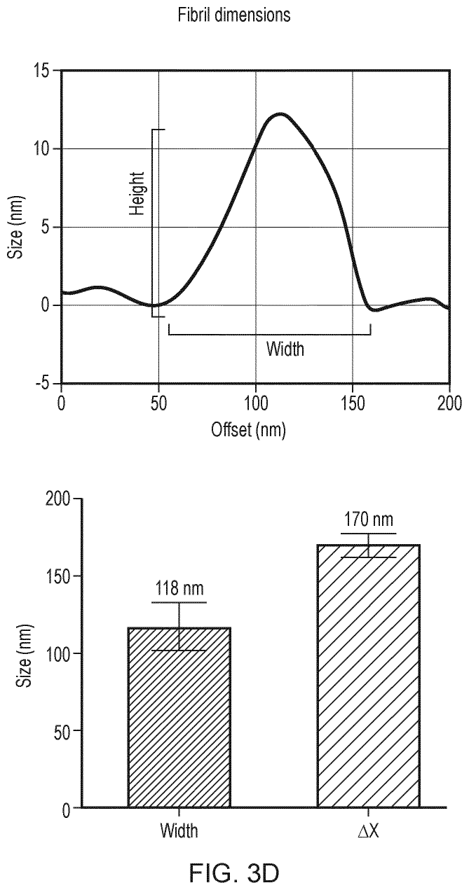

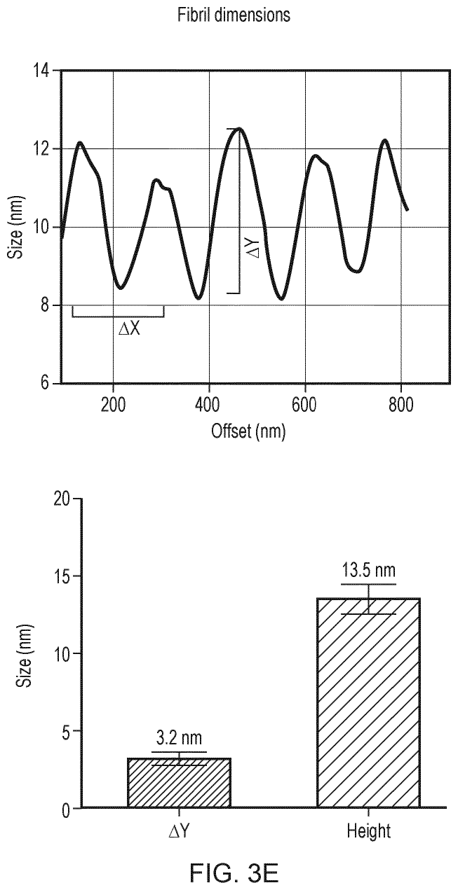

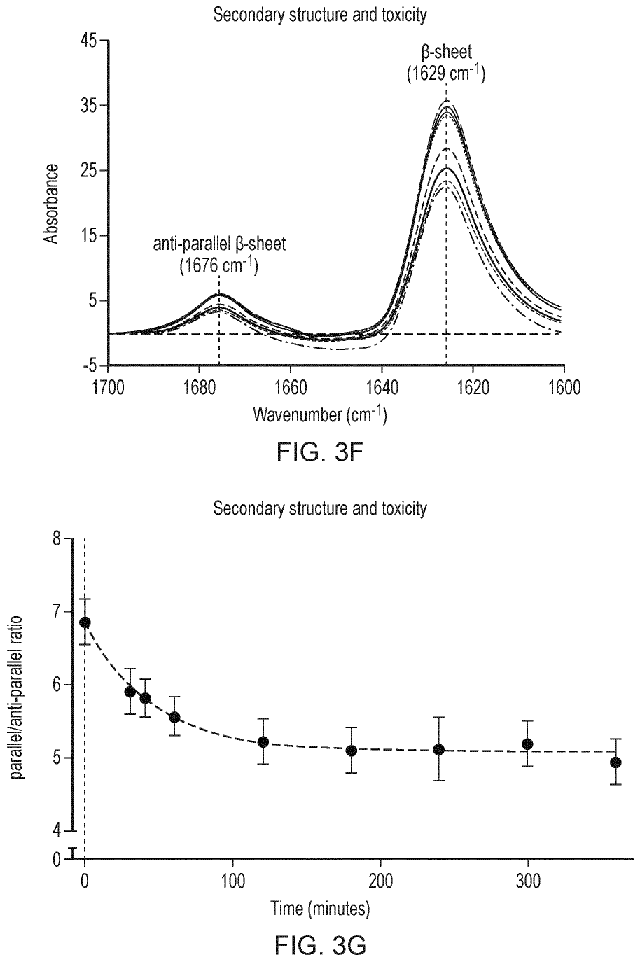

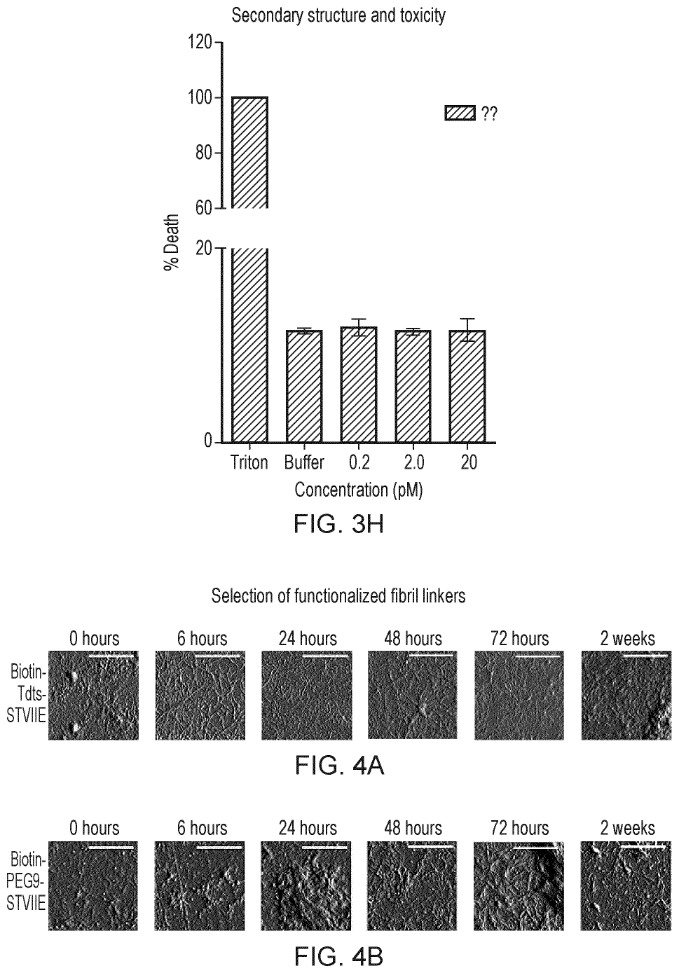

FIG. 3 shows variations on a theme, by employing biotin-PEG13-STVIIE amyloid fibrils. Biotin-PEG13-STVIIE is incubated and allowed to from amyloid fibrils, as shown by (a) AFM, that (b-d) bind streptavidin free in solution, in (e) a concentration-dependent manner, as expected in specific biological interactions.

Immobilizing such fibrils on membranes enables them to be detected in 1 hour, with (f) different concentration of streptavidin derivatized HRP. Then, (g) detection in 30, 60 or 90 minutes of incubation with 1 μg/mL of functionalized enzyme is achieved. Then it is shown that, using (h) 60 minutes of incubation with 1 μg/mL of functionalized enzyme, that 20 ng of biotin-PEG13-STVIIE immobilized amyloid fibrils are detected. The interaction is specific since, in all conditions, pure fibrils of free STVIIE peptide (i.e., not derivatized with biotin) give no signal. This shows that biotin-PEG13-STVIIE fibrils can be used to detect/amplify the presence of immobilized proteins, as demonstrated by (i) immobilizing and detecting the presence of even 2 ng (faint signal) of streptavin-derivatized anti-GFAP antibody. Moreover, BSA protein (negative control, not derivatized) shows no signal, supporting the approach specificity. Experiments conducted in triplicate.

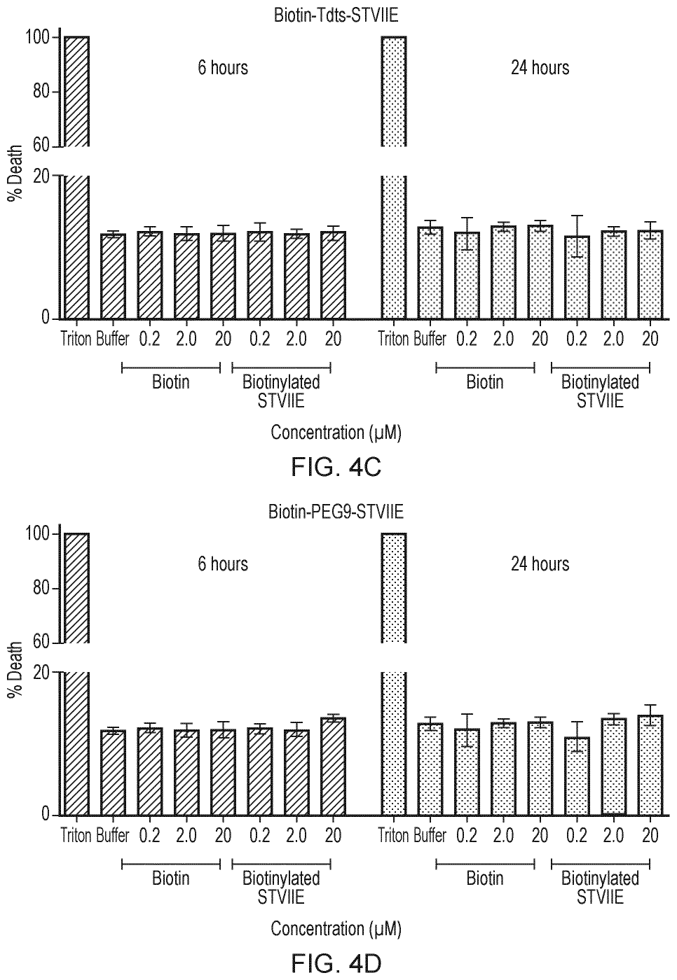

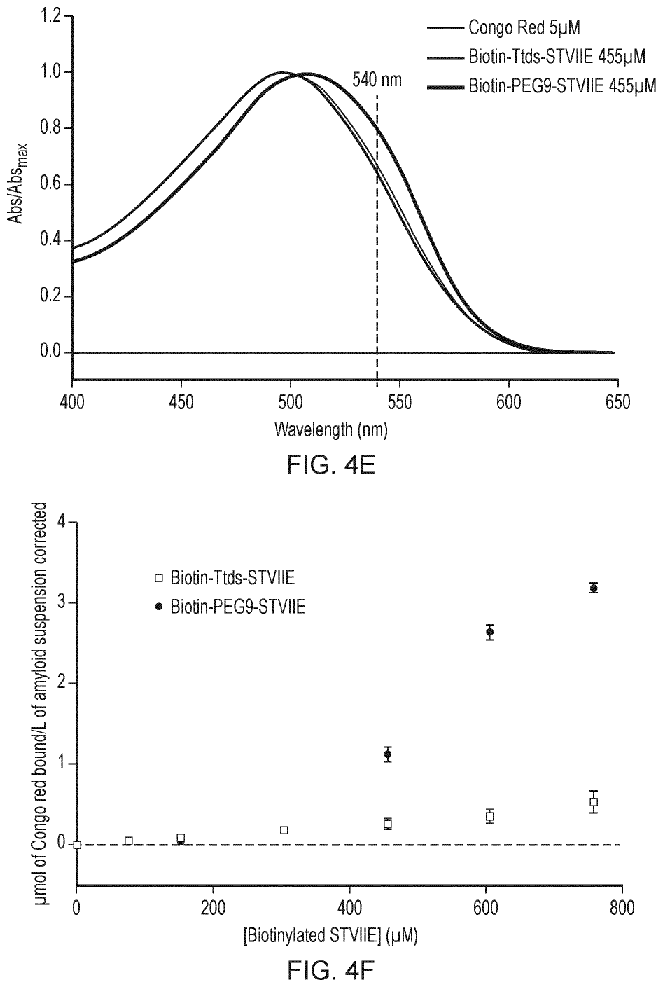

FIG. 4 shows variations on a theme, by employing mixed peptides preparations of amyloid fibrils. Biotin-PEG9-STVIIE peptide monomers are incubated with free STVIIE (not derivatized with biotin) peptide monomers for 2 weeks, at different ratios, and (a) evaluated by AFM, showing fibrils formed after 2 weeks. Biotin-PEG13-STVIIE fibrils are simultaneously evaluated also, as a positive control. Typical (c) FTIR spectra are seen, with (d) peaks consistent with cross beta-sheet structure. If incubated for 2 weeks and, then, (e) immobilized on membranes, preparations containing at time zero 70% free STVIIE and 30% biotin-PEG9-STVIIE (mass percentages of peptide monomers), give an excellent signal after being treated with streptavidin-derivatized HRP. Experiments conducted in triplicate. In addition, immobilizing preparations containing (f) biotin-PEG13-STVIIE peptide monomers incubated with free STVIIE peptide monomers shows that, after one-week co-incubation, such mixed peptides preparations are also easily detectable via dot blot assay. Such arrangement likely results in more biotin moieties available for binding.

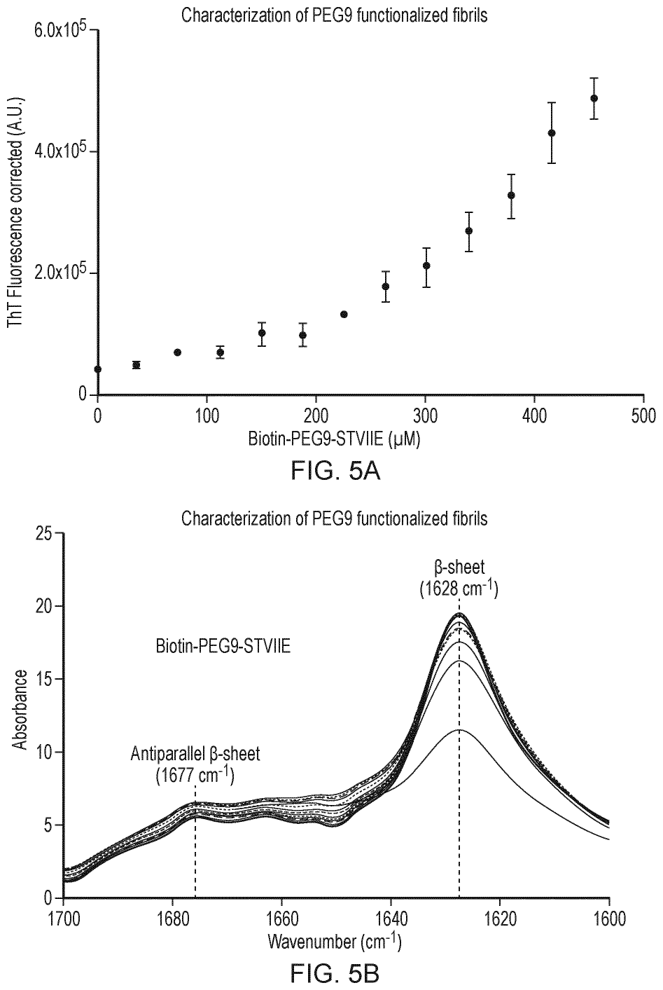

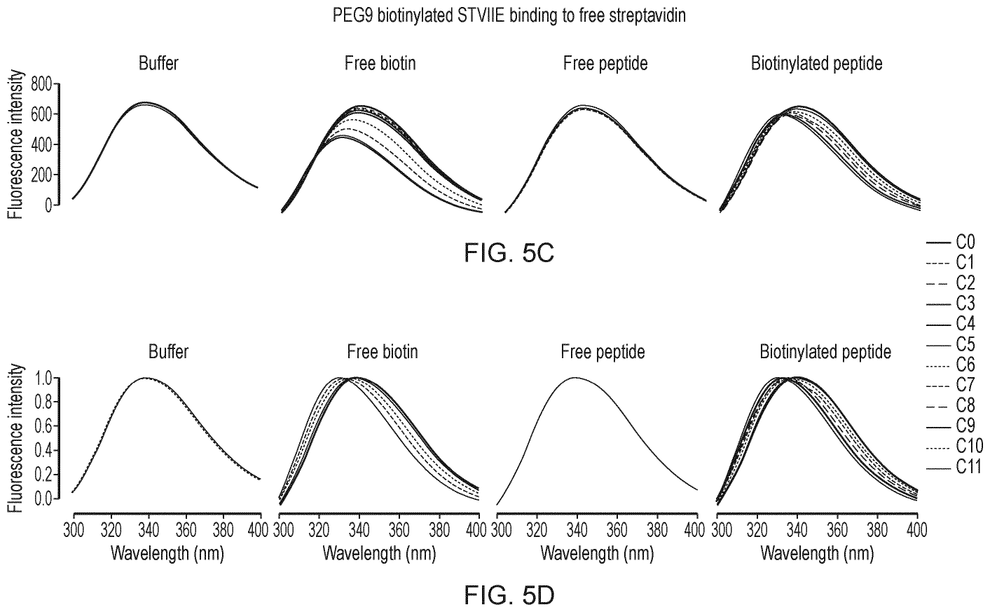

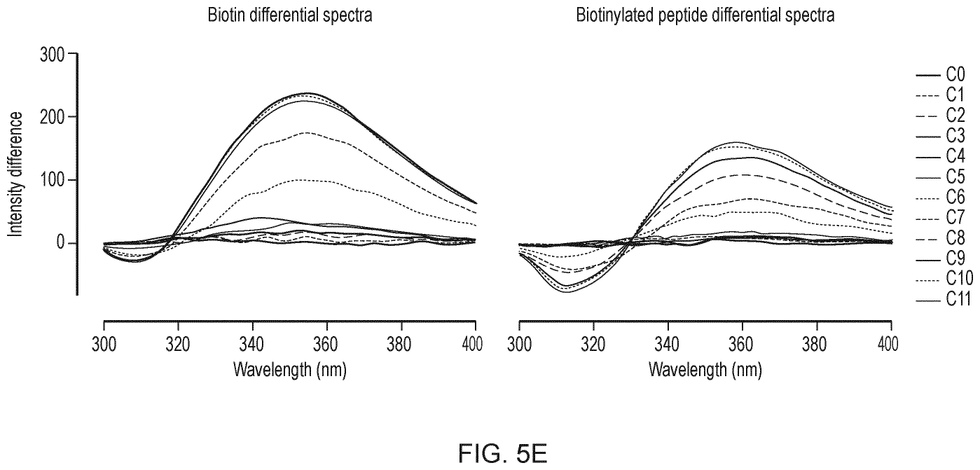

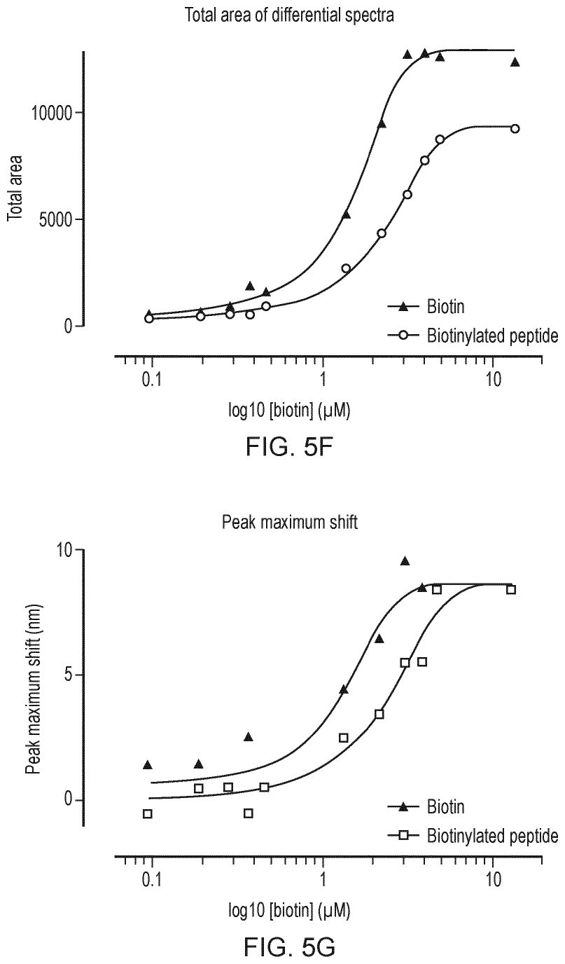

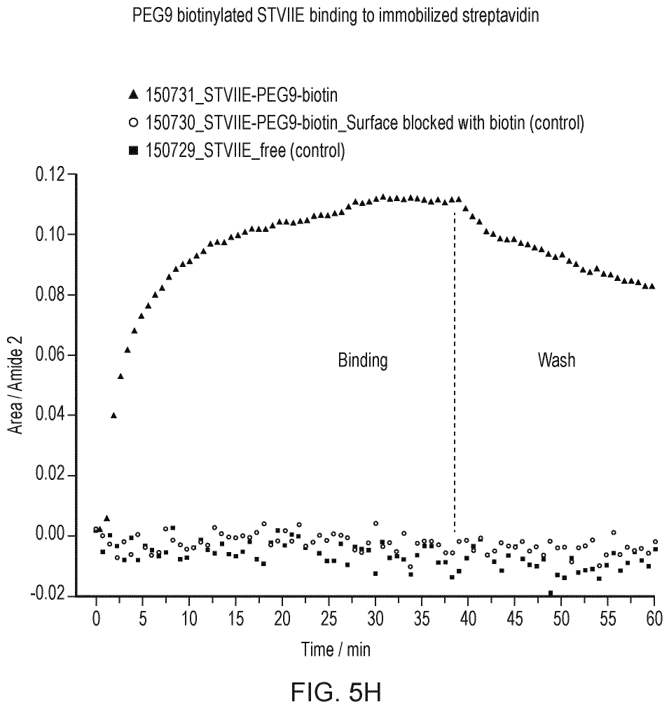

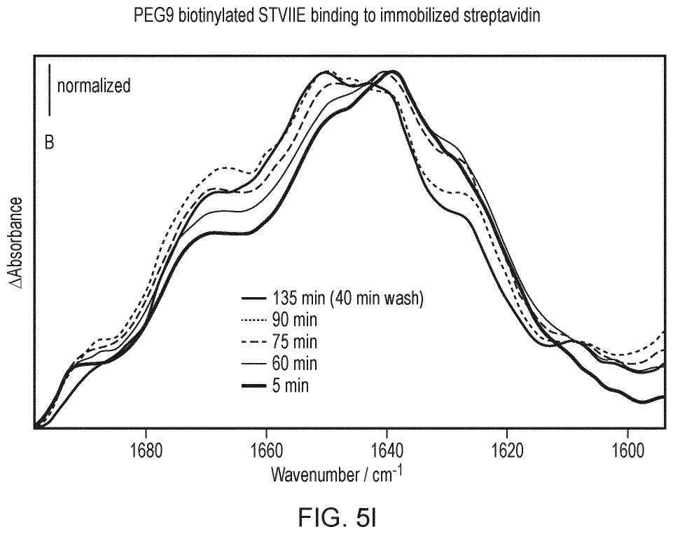

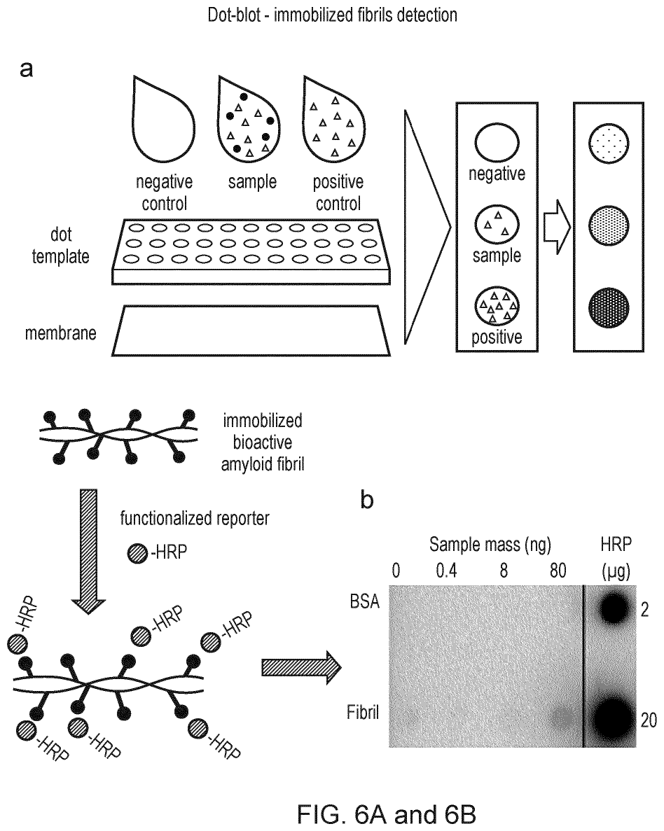

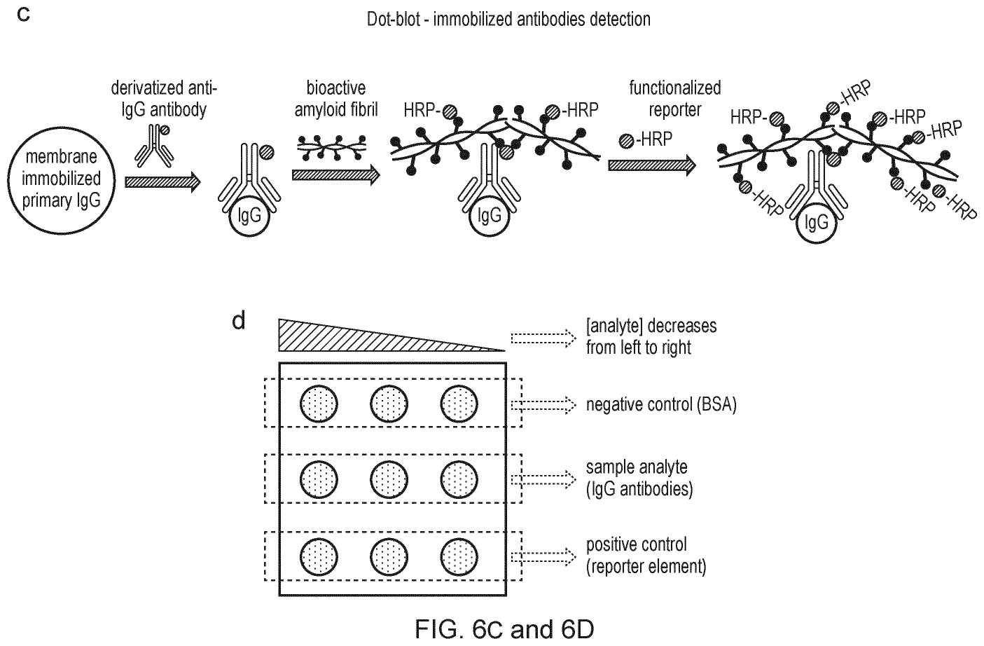

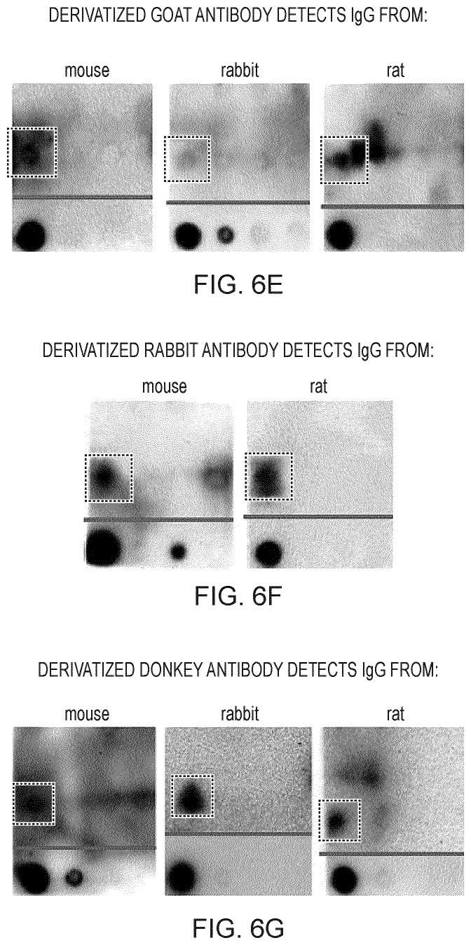

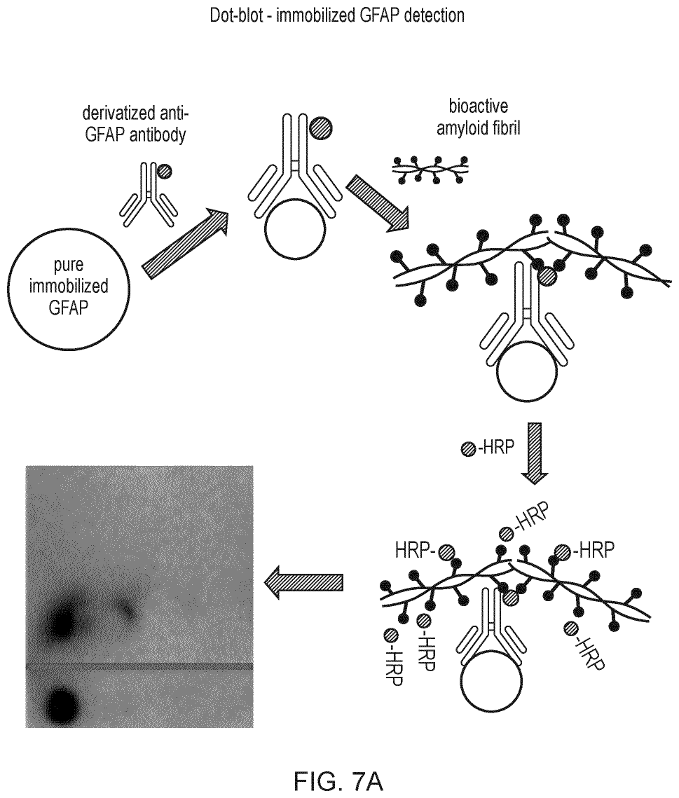

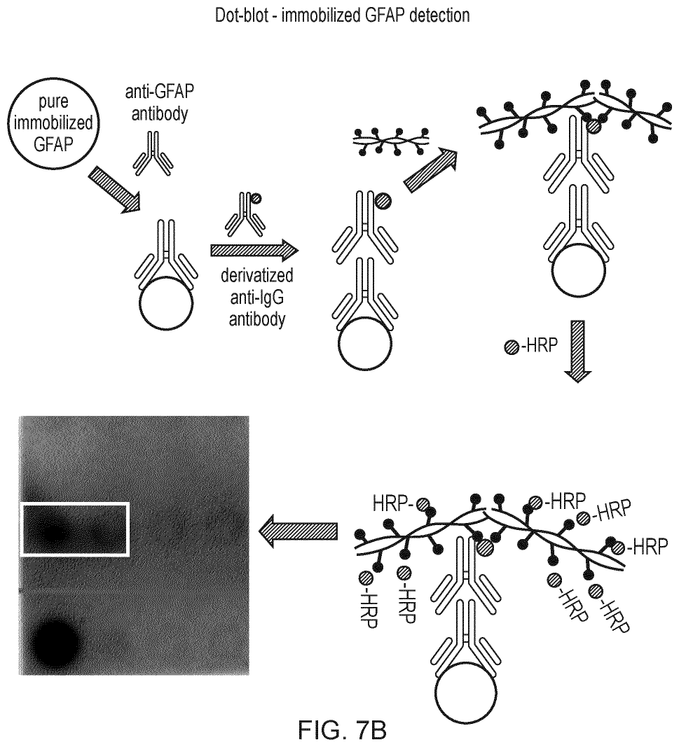

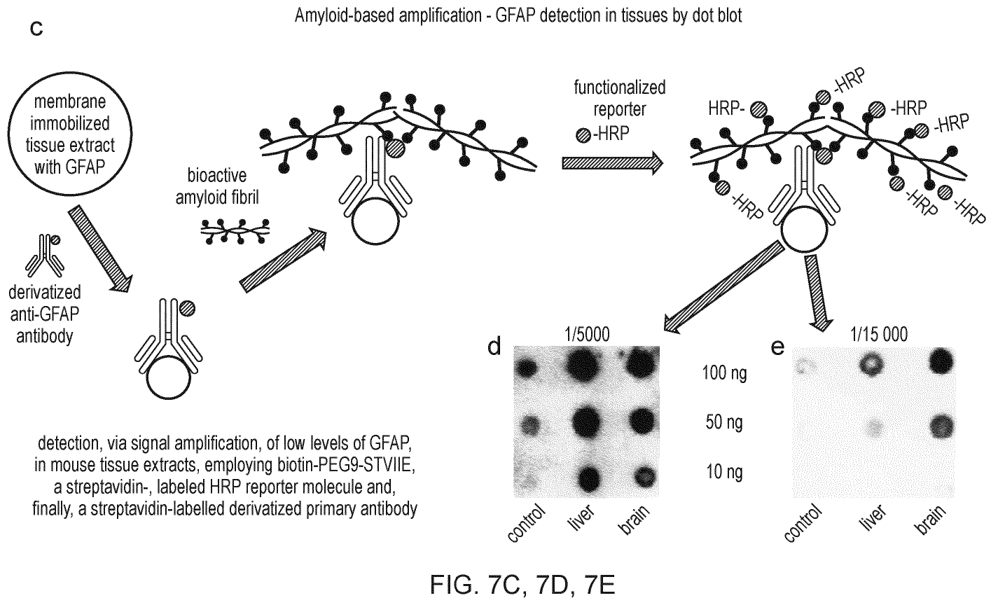

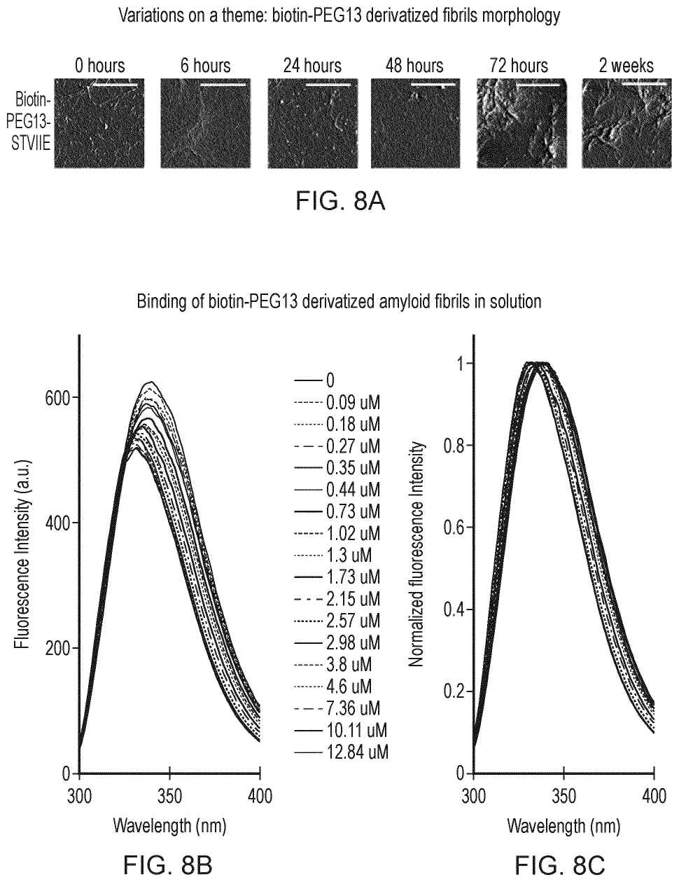

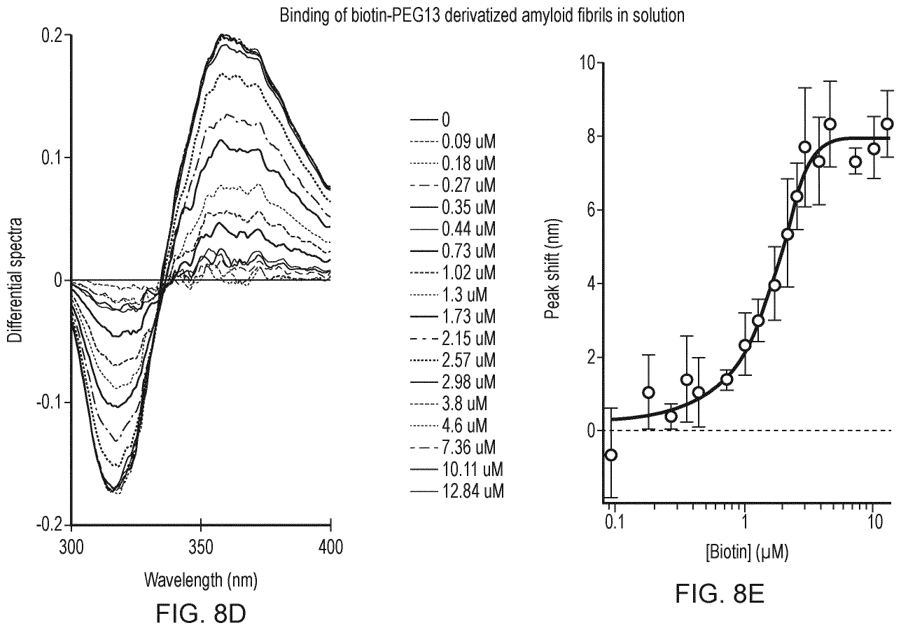

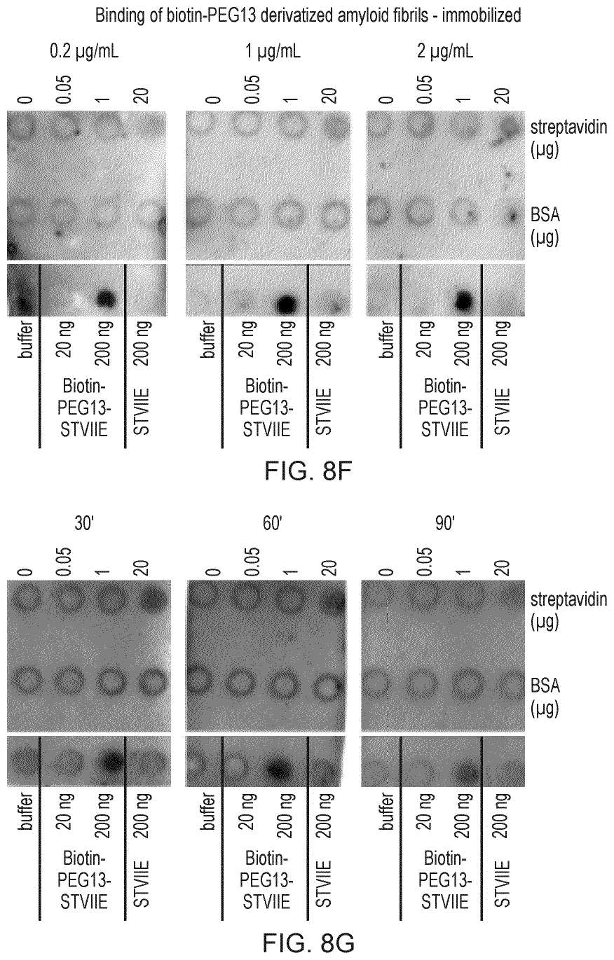

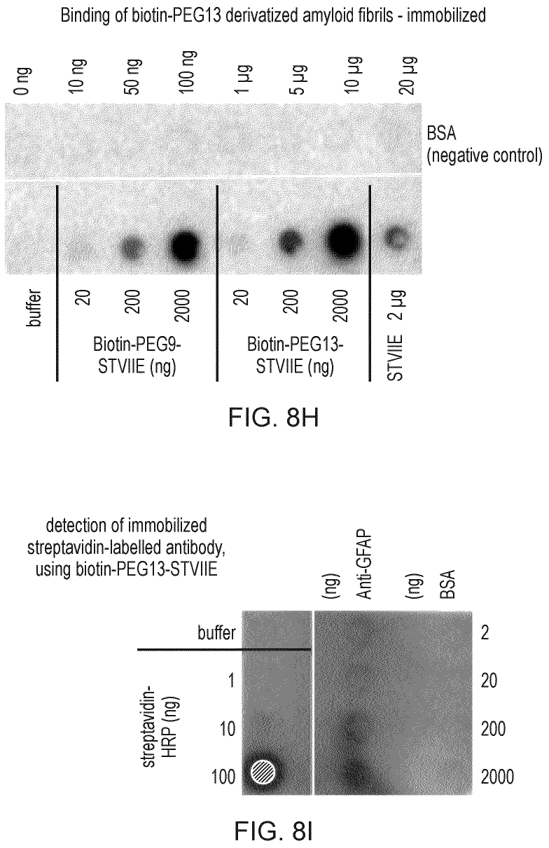

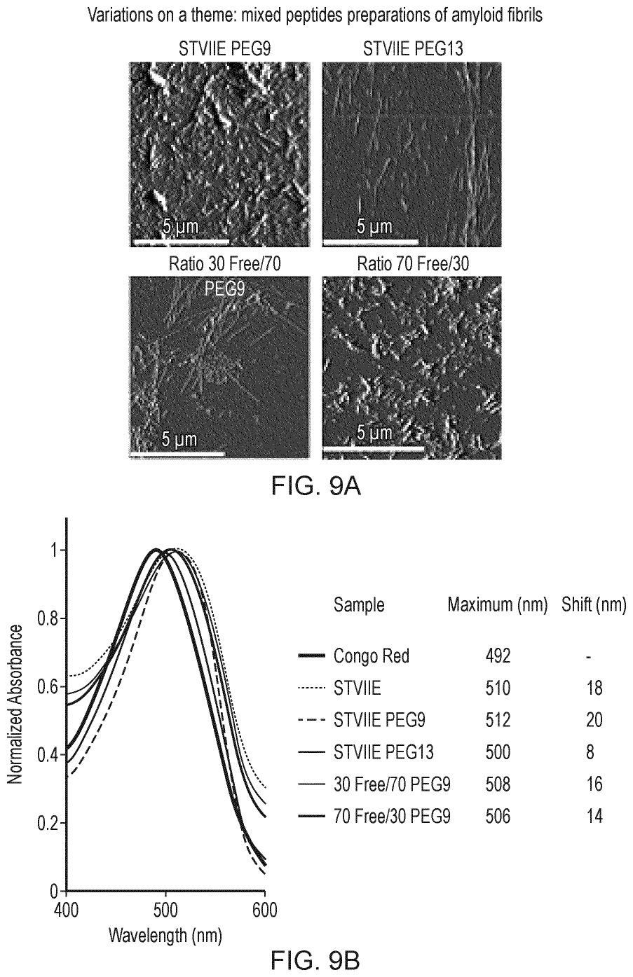

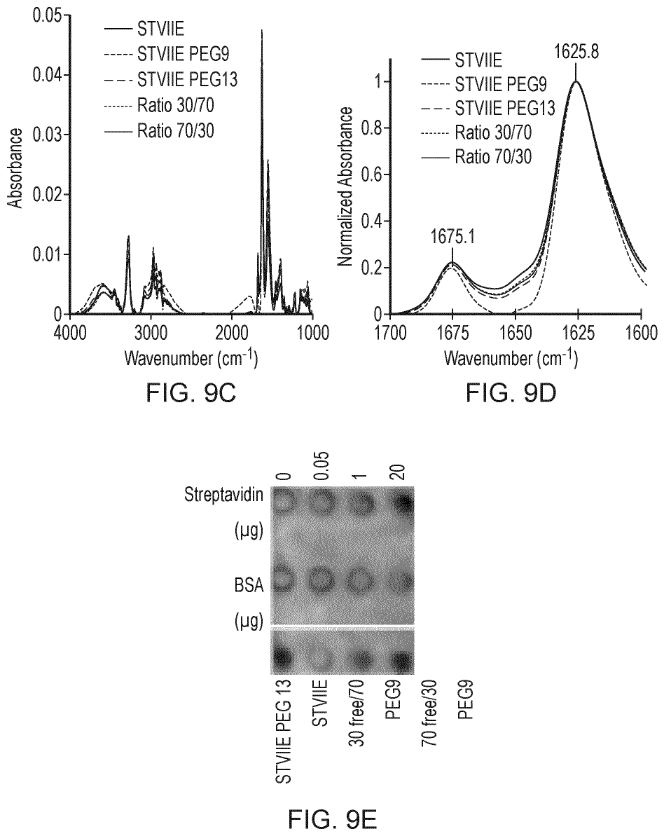

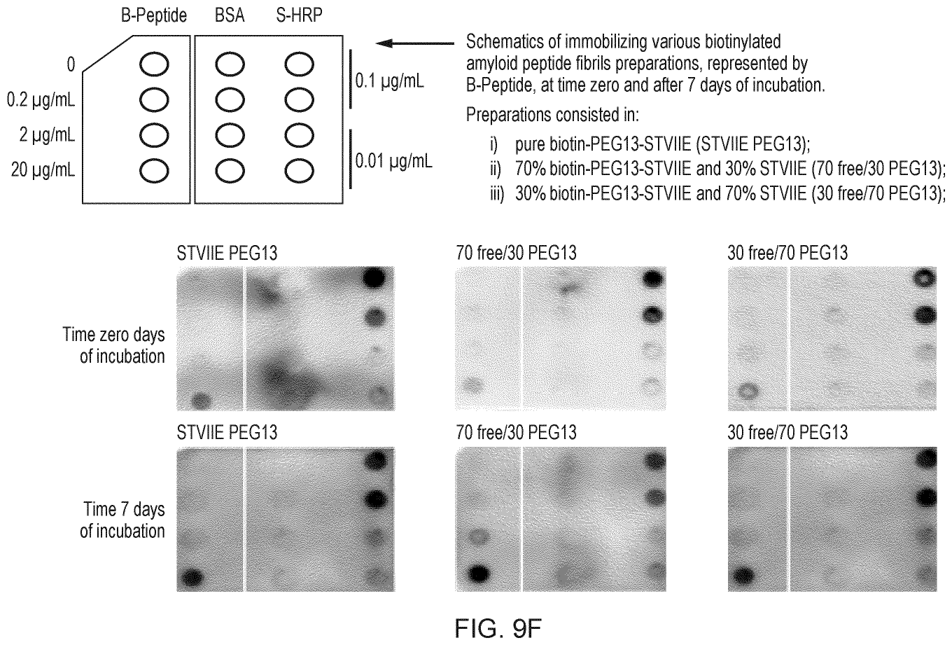

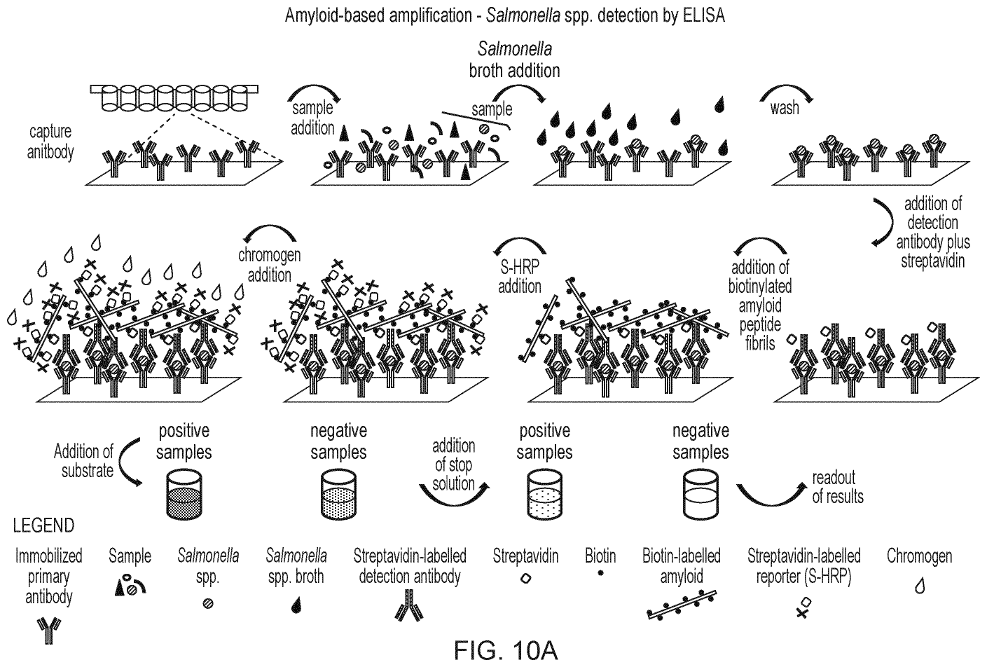

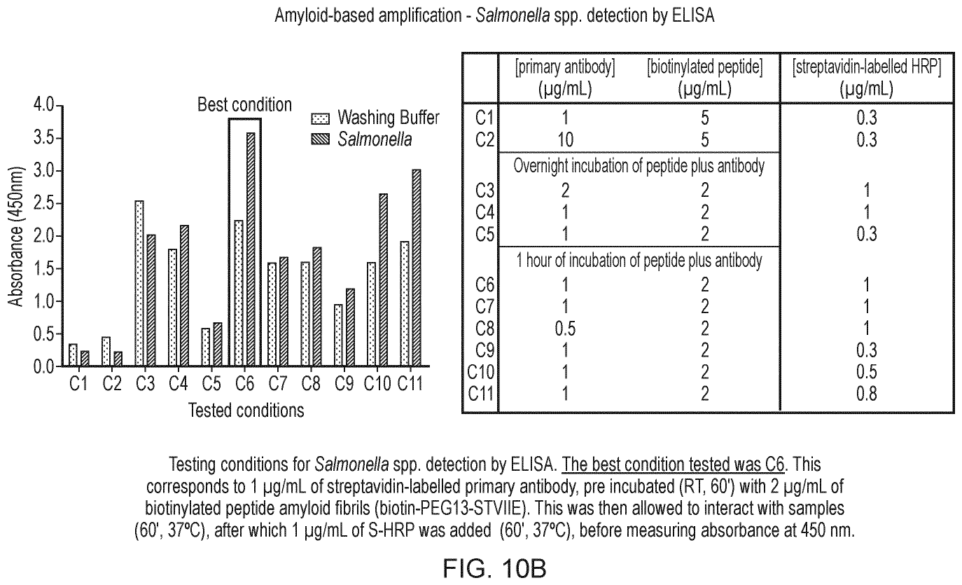

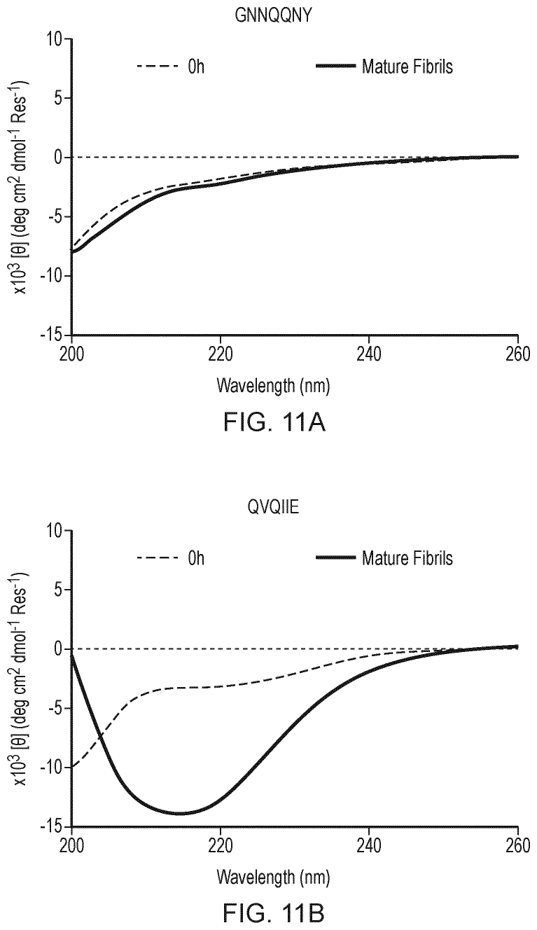

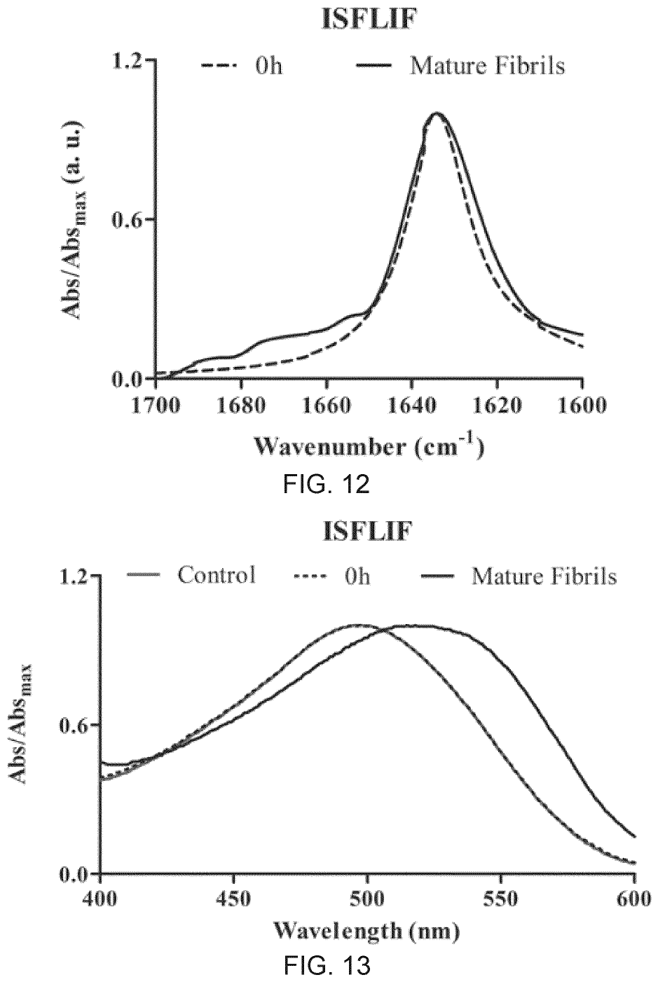

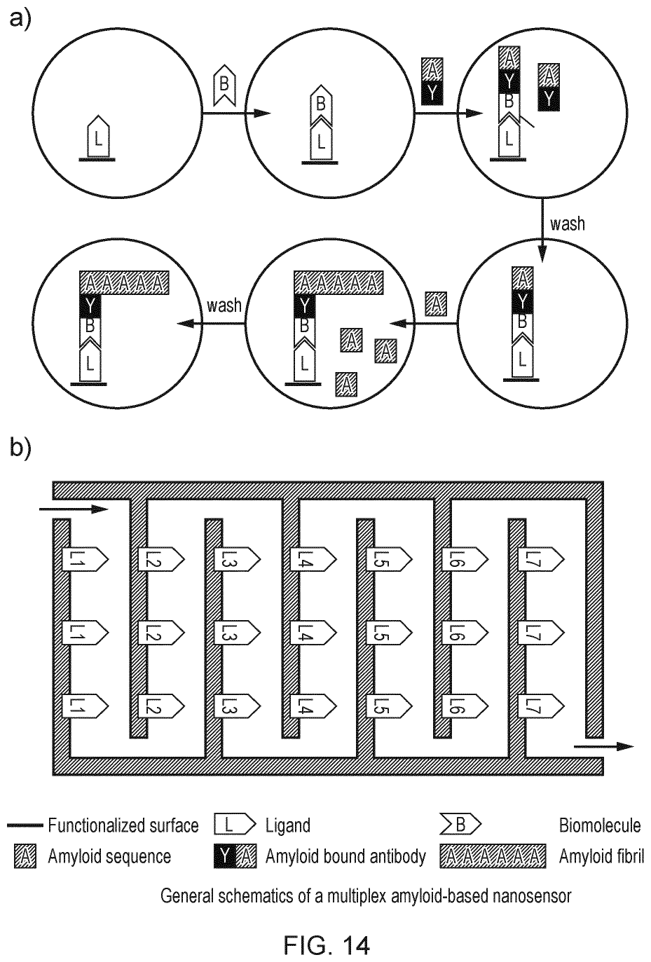

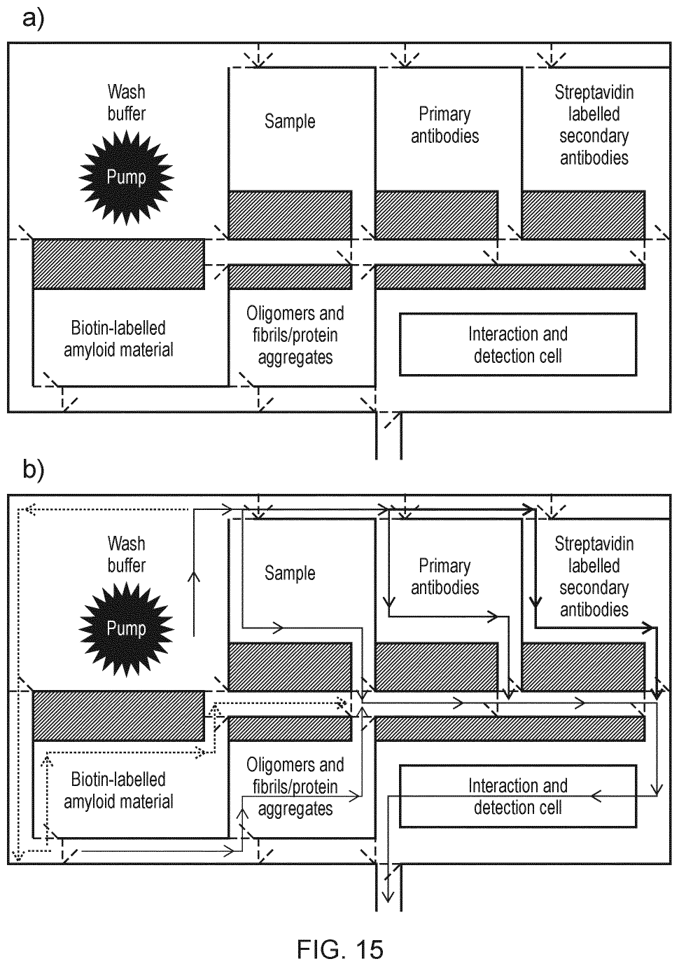

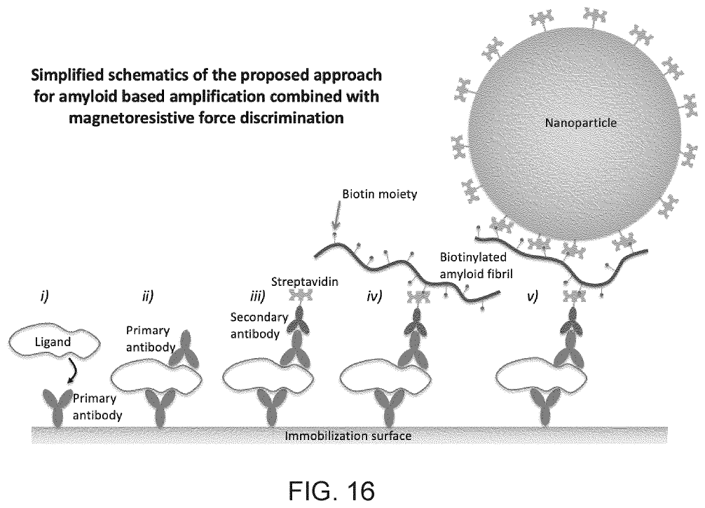

FIG. 5 shows variations on a theme, by detecting Salmonella spp. via amyloid based amplification of ELISA tests. Salmonella spp. detection via amyloid-based amplification in an indirect ELISA test format, as shown in the (a) schematics. The (b) experimental results shown describe 11 conditions tested (varying primary antibody concentration, fibril amounts and also enzyme quantities). The best condition tested was C6. This corresponds to 1 μg/mL of streptavidin-labeled primary antibody, pre-incubated (RT, 60′) with 2 μg/mL of biotinylated peptide amyloid fibrils (biotin-PEG13-STVIEE, allowed to fibrilize for at least 2 weeks). This preparation was then allowed to interact with samples (60′, 37° C.), after which 1 μg/mL of S-HRP was added (60′, 37° C.), before measuring absorbance at 450 nm.

FIG. 11 shows the structural characterization of GNNQQNY and QVQIIE peptide fibrils at physiologic pH conditions. Both peptides GNNQQNY and QVQIIE structure at physiologic pH conditions was determined using CD spectroscopy. (A) CD spectra of the peptide GNNQQNY during incubation at physiologic pH conditions. (B) CD spectra of the peptide QVQIIE during incubation at physiologic pH conditions. Over time the peptide GNNQQNY presents the formation of random coil structures which are not consistent with amyloid fibrils. In more advanced incubation time the peptide QVQIIE evidenced the formation of β-sheet structures that are consistent with amyloid fibrils. For both peptides the CD spectra was acquired at 0 h of incubation (dashed line) and 4 weeks of incubation (continuous lines). Experiments were conducted in triplicate.

FIG. 12 shows structural characterization of peptide ISFLIF at physiologic pH conditions. The structure of the peptide ISFLIF at physiologic pH conditions was determined using FTIR spectroscopy. During incubation the peptide ISFLIF evidenced structures consistent with β-sheet structures, presenting a high band around 1630 cm-1, that is characteristic to a β-sheet conformation (1628-1640 cm-1). All the spectra were normalized and baseline corrected with OPUS Bruker data analysis software (Bruker Corporation, Germany). The FTIR spectra were acquired at 0 h of incubation (dashed line) and 4 weeks of incubation (continuous lines). Experiments were conducted in triplicate.

FIG. 13 shows Congo red interaction with ISFLIF amyloid fibrils formed in physiologic pH conditions. Congo red measurements are commonly used to prove the presence of amyloid fibrils in a sample solution. Using the absorbance of the suspension at 477 and 540 nm it is possible to determine the amount of Congo red bound to amyloid fibrils in suspension, using the equation A540 nm/25 295-A477 nm/46 306. The calculated amount of Congo red bound to amyloid fibrils was corrected to the Congo red contribution. The ISFLIF peptide formed amyloid fibrils in a later stage of incubation as already evidenced by FTIR spectroscopy. The absorbance spectra of the ISFLIF amyloid fibrils in the presence of Congo Red were acquired at 0 h of incubation (dashed line) and 4 weeks of incubation (continuous lines), using the Congo red spectrum (red continuous line) as control. The spectra baseline was corrected in all absorbance spectra acquired. Experiments were conducted in triplicate.

FIG. 14 shows a schematic of a multi-sensing amyloid biosensor as described herein. FIG. 14A shows the assay on a single ligand (“L”). FIG. 14B shows a multiplex biosensor with multiple different ligands (“L1 to L7”).

FIG. 15 shows a schematic of an interaction and detection cell chamber for the assays described herein, and in particular for the multi-sensing amyloid biosensor of FIG. 14. The chamber is shown without (15A) and with the flow of sample and reagents (15B).

FIG. 16 shows a schematic of amyloid based amplification combined with magnetoresistive force discrimination.

### DETAILED DESCRIPTION

The invention is based on a detailed study of amyloid-based biomaterials.

The production and use of a new biomaterial is described herein. The biomaterial has rationally designed and desirable physico-chemical and mechanical properties. In various embodiments, these properties can include at least 1, for example 2, 3, 4 or 5, or more of the following:
a. Resistance to the surrounding milieu, in order for chemical reactions to occur in the immediate vicinity of these biomaterials without affecting them;
b. Can be chemically modified for a specific function, without affecting its chemical and mechanic stability;
c. It self-assembles, typically in a well-established and ordered manner to produce diverse topographies, as needed;
d. It is biologically active, stable and acquires the above properties mentioned in a) to c) in physiological conditions of pH and temperature;
e. It can be derivatized to perform several alternative desired functions, via a set of linker and bioactive moieties connected to it, in a plug-and-play fashion.
f. It is suitable to be used both in gel as well as fibril form, depending on the needs and applications; and/or
g. It is intrinsically biocompatible, non-toxic and of an ultra-small size (compared to conventional materials).

These properties make the biomaterial suitable for a number of uses. In certain embodiments, the biomaterial is useful as a nanomaterial to be integrated in nanodevices for nanotechnology and microfluidics. The invention also provides a method of identifying peptides useful in the formation of biomaterials. Candidate amyloidogenic peptide monomers are tested, to determine those that form amyloid fibrils in physiologic conditions of pH, temperature and ionic strength (at which most physiological protein-ligand interactions occur). Good candidate peptides are (b) functionalized with bioactive molecules bridge elements, via different linkers, and tested regarding their ability to (c) form stable fibrils, that are bound to functional bioactive molecules, connected via functional bridge elements (and adequate linker). Those fibrils that are functional and bioactive are then tested regarding the ability to hold several reporter molecules, both when (d) free in solution and when (e) immobilized.

In some embodiments, the biomaterial is useful for developing and improving diagnostics technologies, for example for signal amplification in immunoassays. In some embodiments, diagnostics technologies allow for the detection of one or more of (i) specific antibodies against: other antibodies, proteins, viruses, bacteria, toxins, hormones, disease (cancer) biomarkers and/or other biomolecules; (ii) peptides and/or proteins (functionalized or not), that can serve to identify the above mentioned targets; (iii) other biomarkers that can be targeted by labelling them with an appropriate linker molecule, as described hereafter.

In certain embodiments, the biomaterial can be functionalised, for example by attaching enzymes and/or other relevant functionalized biomolecules. The biomaterial can hold multiple copies of a given molecule, such as an enzyme and/or antibody, optionally via a linker, bioactive molecules and bridge elements. The biomaterial can, similarly, simultaneously hold several copies of different biomolecules. This property enables the biomaterial to have multiple functions, for example multiple enzymatic and/or biomolecule recognition functions. This allows the biomaterial to be used for biosensing, by using as recognition elements antibodies, DNA, RNA or any other active biomolecule(s) that can be inserted into it.

Other biological activities can be simultaneously engineered into the biomaterial, via the linker, bioactive molecules and/or bridge elements.

Typically, the biomaterial is active both when immobilized as well as when in solution, being also able to interact with molecules active and in solution.

The biomaterial is based on amyloidogenic peptide sequences i.e. sequences associated with or capable of forming amyloid aggregations, fibrils or deposits.

The examples below provide a study of the amyloidogenic properties in physiological conditions of the peptides GNNQQNY, QVQIIE, ISFLIF and STVIIE. These short peptides were previously known to form amyloid fibril structures only in acidic conditions⁴⁹⁻⁵¹. Here we show in particular that STVIIE is able to form fibrils, in physiological pH and temperature conditions. Moreover, it is also demonstrated that these peptides can remain amyloidogenic after being derivatized with biotin, a relevant chemical moiety, widely used in conjugation with streptavidin (to which it binds in an almost covalent manner)^{39,52}. Biotinylated peptide (exemplified by STVIIE) forms amyloid-like structures, which are then demonstrated to be able to bind free in solution and immobilized streptavidin labeled molecules. These are then successfully employed in biosensing. All of the above demonstrate that this amyloid-based technology can be used for signal detection and amplification, among other possible applications.

In a particular embodiment, biotinylated peptide derivatized versions, such as biotinylated STVIIE derivatized versions are shown in the Examples to be particularly useful. Similar results can be obtained with other amyloidogenic protein or peptide sequences, making them equally multi-functional.

Amyloidogenic sequences, such as STVIIE amyloidogenic sequences, derivatized with biotin, via an N-terminal linker, for example a PEG linker such as PEG9 or PEG13, are particularly bioactive. Other linkers can be designed and be highly functional, as described herein. Typically, some flexibility is maintained so that the molecule is not unduly constrained.

Mixed fibril preparations, of biotin-PEG-peptide monomers co-incubated with peptide monomers are also highly functional. For example, mixed fibril preparations, of biotin-PEG9-STVIIE or biotin-PEG13-STVIIE monomers co-incubated with STVIIE peptide monomers are highly functional.

In exemplary embodiments, a mixed fibril preparation may incorporate 60%-90% GNNQQNY, QVQIIE, ISFLIF or STVIIE, preferably STVIIE with 10% to 40% biotinylated peptide (weight/weight), for example 60%-80% GNNQQNY, QVQIIE, ISFLIF or STVIIE, preferably STVIIE with 20% to 40% biotinylated peptide (weight/weight), about 65%-75% GNNQQNY, QVQIIE, ISFLIF or STVIIE, preferably STVIIE with 25% to 35% biotinylated peptide (weight/weight), or about 70% GNNQQNY, QVQIIE, ISFLIF or STVIIE, preferably STVIIE, with about 30% biotinylated peptide (weight/weight). Other amyloidogenic sequences, mixed in similar or different ratios, of free peptide and biotinylated versions, are provided to give a similar result to those demonstrated herein.

The assays described here are performed in physiological conditions. However, if desired, the fibrils' stability allows them also to function in in non-physiological conditions.

In some embodiments, amyloidogenic peptide sequences may be sonicated before use in order to promote fibril formation.

As shown in the Examples below, an amyloidogenic derivatized peptides biomaterial can be incorporated into other technologies and applications, for example diagnostics kits.

The ability of using derivatized amyloidogenic peptides to detect and amplify the presence of several immunoglobulin (IgG) is demonstrated as an example of the utility of this material.

Glial fibrilar acidic protein (GFAP), is detectable, in very low amounts, using several types of derivatized amyloidogenic peptide fibrils that effect signal amplification. Salmonella spp. bacteria are also shown to be detectable, both via a newly-developed assay and when inserted into another commercial immunoassay kit. This detection can be enhanced via signal amplification, resulting in the lowering of the thresholds for accurate diagnostics, for example from days to 6 hours.

The assays are demonstrated via reporter molecule, horseradish peroxidase, that enables a redox (enzyme catalyzed) reaction, that provides the signal. Assays using other labelled reporter molecules, e.g. enzymes, will result in a similar improvement, due to the multiple reporter molecules bound per fibril. Using a redox reaction and a horseradish peroxidase enzyme as reporter molecule, detection, signal amplification, and improved diagnostics are demonstrated via:
a. Indirect ELISA test format (absorbance based readings);
b. Direct ELISA test format (absorbance based readings);
c. Indirect dot blot test format (chemiluminescence based readings);
d. Direct dot blot test format (chemiluminescence based readings);

Detection has been demonstrated with pure samples as well as in tissue extracts (biopsy-like) samples, indicating its clinical applicability.

Given all of the above, the approach and biomaterial(s) are of general applicability to nanotechnology and biomedicine, in the fields of biosensing and beyond.

The biomaterial of the present invention can be used to sample, detect or analyse chemical or biological analytes. A biological analyte may be a biomarker, biological molecule and/or biological fluids. A biological analyte may also be a pathogen, for example a bacteria or virus, as described elsewhere herein.

The biomaterial of invention can be used advantageously to sample biological analytes from a biological sample, such as a biological tissue or a bodily fluid. Biological fluids have typically been excreted or extracted from the body, such as sputum, mucus, saliva, blood, sweat or urine. Other fluids include phlegm, bile, cerebrospinal fluid and amniotic fluid. Ascitic fluid is another typical bodily fluid.

The present invention also provides a method of diagnosing a condition, disease, disorder or irregularity in a subject, said method comprising obtaining a sample of a biological fluid; detecting the presence or absence of a biomarker, biological molecule or metabolite in the sample of biological fluid in an assay using the biomaterial of the invention; and diagnosing the subject based on the presence or absence of the biomarker, biological molecule or metabolite in the biological fluid.

This may be used to detect a biomarker of a disease or disorder, or to detect the presence of a metabolite that is indicative of good or poor health. Alternatively, this method could be used to detect the presence of a narcotic, illicit drug or performance-enhancing drug in the subject.

The analyte may be a hormone or a derived substance thereof. The analyte may be an antibiotic or a derived substance thereof. The analyte may be chemical substance, a narcotic (for example cocaine, heroin, or amphetamine), a performance-enhancing drug (for example a steroid or EPO), an illicit drug, or a pharmaceutical drug.

The analyte may in some embodiments be a toxin, an environmental toxin, a bacterial toxin, or other biologically active molecule.

In some disclosed embodiments, a further step of treating the patient for a diagnosed disease or disorder may be carried out. This may involve a surgical step, or a step of administering a therapeutic agent to a patient in need thereof, at an effective dose.

As used herein, the term “subject” and “patient” includes humans and animals. In certain aspects, the subject is a mammal, for example a rodent, for example a rat, mouse or Guinea pig, a cat, a dog, a goat, a pig, a cow, a horse, or a primate, for example a human. In certain embodiments, the subject is a human. The animal may be a bird. In other embodiments, the subject is a farm animal, for example an ovine animal, a bovine animal, a caprine animal, an equine animal or a bird such as a chicken, turkey, goose or duck. In some embodiments, the analyte to be detected is a biomarker. Biomarkers that are indicative of bacterial infections include cytokines and interleukins. Particular biomarkers include: TNF-related apoptosis-inducing ligand (TRAIL), Granulocyte-macrophage colony-stimulating factor (GM-CSF), Interleukin 1-beta (IL-1β), C-reactive protein (CRP), soluble triggering receptor expressed on myeloid cells 1 (sTREM1), pro-adrenomedullin, serum procalcitonin (PCT), soluble urokinase-type plasminogen activator receptor (suPAR), atrial natriuretic peptide (ANP), IL-6, IL-8, IL-27, and CD64.

Further specific biomarkers that are indicative of viral infections include: Interferon-stimulated gene 15 (ISG15), IL-16, oligoadenylate synthetases-like protein (OASL), Adhesion G protein-coupled receptor E5 (ADGRE5).

Specific cells that are indicative of a disease, disorder or infection to be diagnosed include bacterial cells, including gram-negative bacterial cells, gram positive bacterial cells; host cells such as immune cells, such as dendritic cells, lymphocytes including B cells and T cells, macrophages, NK cells, innate lymphoid cells, eosinophils, basophils, mast cells, neutrophils and/or monocytes; host cells such as cancerous or pre-cancerous cells including, but not limited to, cancer of the respiratory tract such as mouth cancer, tongue cancer, nasal and paranasal sinus cancer, pharyngeal cancer, laryngeal cancer, tracheal cancer, oesophageal cancer, lung cancer, bronchial adenoma; cervical cancer; prostate cancer; colon cancer; rectal cancer; ovarian cancer.

The biomaterial of the invention can in some embodiments comprise a functionalising agent, a bridge element that may optionally be labelled with a reporter molecule, and/or a recognition element that is able to detect a biological molecule, biomarker, protein, virus or cell as described herein. Typically, the bridge element and recognition element each bind to the functionalising agent. Typically, the recognition element comprises an antigen-binding protein, such as an antibody.

In some embodiments, the biomaterial comprises at least the following components:
amyloidogenic peptides of which at least a proportion are functionalised with a biotin functionalising agent;
a streptavidin bridge element labelled with a reporter molecule, bound to at least some of the biotin molecules;
a recognition element formed of a streptavidin-linked antigen-binding protein, such as an antibody, wherein the recognition element will bind to biotin in the biomaterial that is not bound to a bridge element, and wherein the antigen-binding protein binds to a target analyte.

In some embodiments, the antigen-binding protein (e.g. a secondary antibody) may bind to a primary antibody that binds to a target analyte

The term “antigen-binding protein” refers to a protein that is capable of specifically binding an antigen, e.g. a target or its signaling partner, or that is capable of binding an antigen with a measurable binding affinity. Examples of antigen-binding proteins include antibodies or antigen-binding fragments thereof, peptibodies, polypeptides and peptides, optionally conjugated to other peptide moieties or non-peptidic moieties. Antigens to which an antigen-binding protein may bind include any proteinaceous or non-proteinaceous molecule that is capable of eliciting an antibody response, or that is capable of binding to a polypeptide binding agent with detectable binding affinity greater than non-specific binding. The antigen to which a modulating antigen-binding protein binds may include a target, a signaling partner of a target, and/or a complex comprising the target and its signaling partner.

The term “antibody” is used in the broadest sense and includes fully assembled antibodies, tetrameric antibodies, monoclonal antibodies, polyclonal antibodies, multispecific antibodies (e.g., bispecific antibodies), antibody fragments that can bind an antigen (e.g., Fab′, F′(ab)2, Fv, single chain antibodies, diabodies), and recombinant peptides comprising the forgoing as long as they exhibit the desired biological activity. An “immunoglobulin” or “tetrameric antibody” is a tetrameric glycoprotein that consists of two heavy chains and two light chains, each comprising a variable region and a constant region. Antigen-binding portions may be produced by recombinant DNA techniques or by enzymatic or chemical cleavage of intact antibodies. Antibody fragments or antigen-binding portions include, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody, chelating recombinant antibody, a tribody or bibody, an intrabody, a nanobody, a small modular immunopharmaceutical (SMIP), an antigen-binding-domain immunoglobulin fusion protein, a camelized antibody, a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

The term “antibody fragment” herein refers to an antigen-binding fragment of an antibody which retains at least 50% (e.g. at least 60%, 70%, 80% or 90%) of the binding affinity of the entire antibody. When used in a diagnostic-type assay, the antigen-binding protein or antibody used to detect an analyte should be capable of selectively binding to the analyte with greater affinity for the specific biomarker than other molecules present in the same biological fluid. The term “selective” encompasses groups that have an affinity for their target analyte that is more than 2 times greater than for other analytes present in the same biological fluid. For example, the affinity for the target analyte may be 2-10⁹ times greater than for other molecules. In some aspects, the affinity for the target is more than 10 times, 100 time, 1000 times, 10⁴ times, 10⁵ times, 10⁶ times, 10⁹ times greater for the target than for other molecules in the same biological fluid.

As used herein, a “biomarker” is a characteristic that can be objectively measured and evaluated as an indicator of normal biological processes, pathogenic processes, or pharmacologic responses to a therapeutic intervention. Biomarkers may be cells, biological molecules such as proteins, lipids, hormones and/or nucleic acids. The term “biomarker” as used herein include biological molecules and metabolites. The biomarker may be a class of biomarkers, such as proteins, lipids, cells, hormones and/or nucleic acids. The functional group may bind selectively to the entire class of biomarker or may bind to a subset of the class of biomarker. For example, when the biomarker is a peptide, the functional group may bind to all peptide, to specific classes of peptides such as interferons, immunoglobulins, or cytokines; or to individual peptides such as interferon α, interferon β, interferon γ, CRP, TRAIL, sTREM-1, procalcitonin, ANP, pro-vasopressin, proadrenomedullin, suPAR, lactoferrin, galectin-9, CD14, CD32, CD35, CD46, CD55, CD59, CD64, CD88, interleukins including IL-1, IL-4, IL-6, IL-8, IL-10, IL-12, IL-17, IL-27. In a further example, when the biomarker is a cell, the functional group may bind to all cells, or to a specific class of cells such as: bacterial cells, including gram-negative bacterial cells, gram positive bacterial cells; host cells such as immune cells, such as dendritic cells, lymphocytes including B cells and T cells, macrophages, NK cells, eosinophils, basophils, neutrophils and/or monocytes; host cells such as cancerous or pre-cancerous cells including, but not limited to, cancer of the respiratory tract such as mouth cancer, tongue cancer, oesophageal cancer, lung cancer; cervical cancer; prostate cancer; colon cancer; rectal cancer, ovarian cancer.

The biomaterial also finds particular utility in the detection of pathogens. Typically, the recognition element will be able to bind specifically to a pathogen.

Typical pathogenic bacteria for detection may be gram negative or gram positive bacteria. The bacteria may be cocci such as Staphylococci, Streptococci (e.g. S. pneumonia) or Neisseriae (e.g. N. gonorrhoeae or N. meningitidis), gram positive bacilli such as Corynebacteria, Bacillus Anthracis, Listeria monocytogenes, gram negative bacilli such as Salmonella spp., Shigella, Campylobacter, Vibrio, Yersinia pestis, Pseudomas spp., Brucella, Haempohilus, Legionella or Bortedella. Other bacteria that can be detected include Mycobacteria such as M. tuberculosis, M. leprae or M. avium, Rickettsia, or Chlamydia.

In some embodiments, the pathogen may be a virus. Typical viruses that can be detected include: DNA viruses such as adenovirus, herpesvirus, poxvirus, parvovirus, papilloma virus or hepatitis, for example hepatitis B; or RNA viruses such as influenza, coronaviruses, paramyxovirus, picornavirus (e.g. polio, coxsackie, hepatitis A, rhinovirus), togaviruses (e.g. rubella), flaviviruses (e.g. causing yellow fever, dengue fever), rhabdoviruses (e.g. rabies), ebolavirus, or retroviruses such as HIV.

Pathogenic fungi that can be detected include Candida albicans, Aspergillus or Pneumocystis.

Pathogenic protozoa that can be detected include Leishmania, Plasmodium, Trypanosoma, Toxoplasma gondii or Crytosporidium.

In certain embodiments, the pathogen to be detected is a rhinovirus, coronavirus, influenza virus, adenovirus, or respiratory syncytial virus. In an exemplary embodiment, the pathogen to be detected is a coronavirus, more typically a human coronavirus such as the 2019-nCOV.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### EXPERIMENTAL

Detection of Glial Fibrillar Acidic Protein and Other Proteins as an Example of Detection Using Amyloid-Based Biomaterials

Amyloid fibrils are formed via the amyloidogenesis process, by which peptide or protein monomers aggregate into higher order aggregates. Although amyloid fibrils are often associated with human degenerative pathologies (such as Alzheimer's and Parkinson's diseases), they can perform physiological roles. Moreover, amyloids have also been suggested as potential novel biomaterials. Short amyloidogenic peptide sequences that form stable fibrils in physiological conditions are ideal for nanotechnology and nanomedicine, for example as bioactive gels and/or biosensing platforms. For that, amyloidogenic molecules must be able to be functionalized with a relevant chemical group, for example biotin. Biotin binds to streptavidin (and its homologous avidin), forming the strongest protein-ligand non-covalent interaction in Nature. For this reason, this interaction system has been widely used in many applications, both as initial proof-of-concept and in fully mature technologies.

Here we describe a new technology, based in the production, from short amyloidogenic peptides, of stable amyloid fibrils at physiological pH and temperature conditions.

As demonstrated in the non-limiting experiments reported below, these stable fibrils can be derivatized with biotin and are functional and able to bind streptavidin, both when the later is free in solution and when it is immobilized on a surface.

Subsequently, these functional fibrils are employed in a proof-of-concept experiment to detect immobilized Glial Fibrilar Acidic Protein (GFAP) in tissue samples, in a dot blot immunoassay. Briefly, GFAP detection and amplification is enabled by streptavidin-labeled primary antibodies and our biotin-labeled peptide fibrils, to which streptavidin-labeled reporter molecules later bind. After that, a similar approach was applied with streptavidin labeled secondary antibodies, raised against different Immunoglobulin G (IgG) primary antibodies, immobilized in a dot blot assay. Several combinations of animal species as sources of primary and secondary IgG antibodies were used, showing the general applicability of the approach.

Finally, with another specific target, Salmonella spp., we employed this amyloid-based technology for the target detection in both a dot blot and a standard enzyme linked immunosorbent assay (ELISA) formats, demonstrating its applicability in yet another model system.

All of these experimental results demonstrate, in different detection, amplification and immunodiagnostics assay formats, the general applicability of the technology.

Materials and Methods

Chemicals

The peptides used (STVIIE, GNNQQNY, QVQIIE and ISFLIF) were purchased from JPT Peptide Technologies (JPT Peptide Technologies GmbH, Berlin, Germany) with a purity of 95% while Aβ(1-42) peptide was purchased Phoenix Pharmaceuticals Inc (Phoenix Pharmaceuticals, Inc., California, USA) with a purity over 97%. Congo red was purchased in Sigma (Sigma-Aldrich Quimica, S. L., Sintra, Portugal). N-terminus biotinylated STVIIE were also commercially obtained from JPT Peptide Technologies (JPT Peptide Technologies GmbH, Berlin, Germany). Tris (tris(hydroxymethyl)aminomethane) and EDTA (ethylenediaminetetraacetic acid) were purchased from Merck (Merck KGaA, Darmstadt, Germany) and Sigma (Sigma-Aldrich Quimica, S. L., Sintra, Portugal), respectively.

Sample Preparation

STVIIE, GNNQQNY, QVQIIE and ISFLIF were prepared at the highest final concentration that they were able to be fully dissolved, respectively 1 mg/mL (STVIIE and GNNQQNY), 0.55 mg/mL (QVQIIE) and 0.1 mg/mL (ISFLIF). For all peptide samples the final incubation buffer was 50 mM Tris-HCl pH 7.5, 5 mM EDTA buffer. Briefly, after weighing the peptide, half of the final volume of H₂O ultrapure was added. A short vortex of approximately 1500 rpm and 30 seconds and an ultrasound bath of 280 seconds in water/ice were performed two times. The other half of the final volume was then added, containing 100 mM Tris-HCl pH 7.5, 10 mM EDTA buffer was added, with the peptide samples being therefore in the final incubation buffer. Before incubation, two other cycles of vortex and ultrasound bath were performed. The final peptide solution sample was incubated at room temperature.

Circular Dichroism Spectroscopy

Following previous approaches^{11,53}, circular dichroism (CD) measurements were carried out in a JASCO spectropolarimeter J-815 (Tokyo, Japan), using cuvettes of 1.0 mm path length. Spectra were acquired between 195 and 260 nm, at 25.0° C., with data pitch of 0.5 nm, wavelength sampling velocity of 200 nm min⁻¹, data integration time of 1 s and performing at least 3 accumulations. Measurements were conducted in 50 mM Tris-HCl pH 7.5, 5 mM EDTA buffer. In addition to blank subtraction, experimental instrument-related baseline drift was corrected by subtracting to all spectra the average of the signal between 250 and 260 nm. The spectra were normalized to mean residue molar ellipticity (deg cm² dmol⁻¹ residue⁻¹). All conditions were measured independently and in triplicate.

Fourier Transform Infra Red (FTIR) Spectroscopy

Following previous approaches^{11,54}, peptide samples were applied to the FTIR sample holder at 298.15 K. InfraRed spectra were recorded on a Bruker Tensor 27 infrared spectrophotometer (Bruker Optik GmbH, Ettlingen, Germany) equipped with a Bio-ATR II accessory. The spectrophotometer was continuously purged with dried air. Spectra were recorded at a spectral resolution of 4 cm⁻¹ and 120 accumulations were performed per measurement. The final spectra were corrected to the baseline (the final incubation buffer) and rescaled in the amide I area (˜1600 to ˜1700 cm⁻¹).

Congo Red Assay

In line with previous work^{55,56}, before measurements, peptides were diluted ten times from their stock incubation conditions in 50 mM Tris-HCl pH 7.5, 5 mM EDTA buffer and incubated with κ μM Congo red for 1 hour. The readings were performed using a UV-Vis Spectrophotometer Shimadzu UV-2700 (Shimadzu Corporation, Kyoto, Japan) with a wavelength between 300 nm and 700 nm. Readings were recorded in triplicate. The fibrillation kinetics was recorded in triplicate for a total period of 4 weeks of incubation.

Atomic Force Microscopy

According to experimental procedures at the host lab⁵⁷⁻⁵⁹, the samples were placed in poly-1-lysine slides and dried using a vacuum chamber. When fully dried, the samples were rinsed with ultra-pure water and dried with a gentle N₂ air flux. A NanoWizard II atomic force microscope (JPK Instruments, Berlin, Germany), mounted on the top of an Axiovert 200 inverted optical microscope (Zeiss, Jena, Germany), was used for the microscopic experiments. The AFM head is equipped with a 5.85-μm z-range linearized piezoelectric scanner and an infrared laser. For the acquisition of the different AFM images, it was used a ACL-50 tip (Applied NanoStructures, Inc., California, USA) with a spring constant between 20-95 N/m and a frequency between 145-230 kHz. Amyloid fibrils were acquired in intermittent (air) mode with a setpoint of between 0.4-0.5 V, a line rate of 0.7-0.8 Hz, an IGain of 20-50 Hz and a PGain of 0.001-0.004. The images acquired were treated afterwards with the JPKSPM Data Processing (JPK Instruments, Berlin, Germany).

Morphological Characterization of the Amyloid Fibrils

The morphological characterization of the AFM images of ISFLIF amyloid fibrils with and without biotin was carried out in the program Gwyddion 2.31. Using the extract profiles command cross lines were drawn in the fibrils surface allowing the determination of the fibrils height and width. The height and width values determined resulted from the average of 20 individual fibers from at the least three different fibrils AFM images. The height and width values were presented as the mean with the associated standard error of the mean (SEM).

Cell Culture and Cytotoxicity Assays

Human H4 neuroglioma cells were maintained at 37° C. in OPTI-MEM I (Gibco, Invitrogen, Barcelona, Spain) supplemented with 10% fetal bovine serum and seeded onto 24-well plates at a density of 60.000 cells/cm² 24 h prior treatment. Cells were treated with 0.2, 2 and 20 μM of fibrillated STEVIIE, its biotin conjugated species, biotin and vehicle for 6 and 24 hours. Conditioned media of treated cells was collected, and cytotoxicity immediately assessed by measuring the activity of released lactate dehydrogenase (LDH) in a plate reader (Tecan Infinite 200), according to the manufacturer's protocol (Clontech). The maximum activity was determined by lysing the cells with triton X-100 (final concentration 1%).

Dot-Blot Assays

A vacuum-based Dot Blot 48-sample apparatus is applied in the dot blot technique to immobilize samples onto the membrane. A polyvinylidene difluoride (PVDF) membrane is activated by submersion in methanol for 30 sec and then washed in distilled water and saline buffer to remove methanol. Samples are diluted in a saline buffer and immobilized on the PVDF membrane. Membrane is then blocked with 5% of bovine serum albumin at room temperature for 60 min in the 2D rotator in order to decrease non-specific binding. After blocking, three wash cycles are performed for 10 min each, the first two with a saline buffer plus a mild detergent and the final cycle with saline buffer alone. Depending on the purpose of the dot blot assay, antibodies are incubated with DART technology for further samples detection. The reporter molecule used is HRP conjugated to streptavidin. Finally, the substrate Clarity™ ECL is incubated with the membrane and then metabolized by the enzyme. A dot-shaped visible signal is produced and detected by chemiluminescence, indicating a positive result, using the Bio-Rad ChemiDoc™ XRS+ apparatus.

Results

A peptide that forms fibrils with stable amyloid morphologies in physiological conditions of pH and temperature is preferred, in order to be compatible with most biologically relevant protein-ligand and antibody-ligand interactions. It is important to have a short peptide sequence (to keep costs low) that readily dissolves in water (instead of hydrophobic solvents) at high concentrations. If stable fibrils are formed it is then possible to develop nanotechnology applications, namely biomolecule detection and/or signal amplification. The approach scheme is shown in FIG. 1.

The approach starts from ideal peptide sequences (known to be amyloidogenic) and tests their ability to form amyloid fibrils in physiological conditions (FIG. 1a). Test(s) with the most promising sequence(s) are then conducted, to characterize amyloidogenic behavior, morphology, size and toxicity. This is done both with native amyloid sequences and sequences functionalized with bioactive chemical moieties (FIG. 1b). Then, the most promising functionalized sequences are selected, namely those that, if derivatized with relevant molecules, remain able to form stable fibrils. These are tested regarding the ability to bind/interact with the bioactive molecule (FIG. 1c). Essentially, at this stage, the objective is to confirm that the amyloid fibril into which a novel biological function was inserted (via the addition of a chemical moiety) preserves the amyloid features and is functional. Having a bridge element connected to the fibril allows it to bind multiple partners, which provides a particular advantage because a reporter molecule can then bind to this bridge element. Each fibril can have multiple copies of the bridge element and it will thus bind multiple reporter molecule copies (FIG. 1d). Subsequently, classical biomolecule recognition can take place by employing antibodies functionalized to bind the bridge element. This leads to multiple reporter reactions for each single antibody-ligand interaction, resulting in increased signal detection and amplification (FIG. 1e).

Biotin was used to functionalise amyloid fibrils, streptavidin used as bridge element, horseradish peroxidase (HRP) linked to streptavidin as reporter molecule, and streptavidin-labeled primary antibodies as recognition elements. Variations of this approach may be employed, with other bridge elements, reporter molecules and/or recognition elements (in particular other antibodies and derivatized DNA or RNA detection sequences, to identify and target other specific biomarkers). These results provide proof-of-concept, establishing the feasibility of the technology.

The most promising amyloidogenic peptide is typically selected. For this purpose, atomic force microscopy (AFM) was employed. AFM is a technique that can be very useful in the acquisition of surface images with high resolution and sensibility, being therefore suitable for such studies of amyloid fibrils and employed in this analysis. Representative AFM images typical of each sample are displayed in FIG. 2. Regarding GNNQQNY (FIG. 2, first row from the top), at the start of incubation there are no visible structures. At the end of the incubation period tested (2 weeks), heterogeneous structures are visible. These are not consistent with the well-defined amyloid fibril morphology, in agreement with the CD data suggesting random coil structures (FIG. 11). In contrast, QVQIIE, ISFLIF and STVIIE peptides show amyloid morphologies at the end of the incubation period tested (FIG. 2). Of these, STVIIE displays a typical amyloid morphology, as discussed ahead. QVQIIE morphology is consistent with an amyloid-like gel structure while ISFLIF also displays classical amyloid fibril morphology. This is consistent with the β-sheet content of QVQIIE and ISFLIF at the end of the incubation period, measured via Fourier transform infrared spectroscopy (FIG. 12) and binding profile to Congo Red (FIG. 13). However, ISFLIF is difficult to dissolve at high concentrations in physiological conditions. As for STVIIE, it forms clear fibril structures in the conditions tested. It also starts forming them at an earlier time point (about 6 hours) than ISFLIF and is soluble at high concentrations (1 mg/mL), both important properties for detection and signal amplification. STVIIE is thus employed in the nanotechnology applications described here, at physiological pH and temperature. It is used in to develop the signal amplification method, as described ahead.

STVIIE amyloid fibril formation process in physiological conditions of pH and temperature was further characterized (FIG. 3). AFM allows nanoscale analysis of STVIIE amyloid fibrils (FIG. 3a, first five leftmost images), at several incubation times: 0 h, 6 h, 24 h, 72 h and 2 weeks. Throughout incubation there is a visible increase in amyloid-like fibril structures. Amyloid β peptide (involved in Alzheimer's disease), Aβ(1-42), was incubated for two weeks and used as a positive control of fibrillization, while buffer and empty slides were employed as negative controls of AFM experiments (FIG. 3a, three rightmost images). As expected, negative controls do not form fibrils and the Aβ(1-42) positive control form typical amyloid fibrils.

Regarding STVIIE amyloid fibrils, it is clear that they display repetitive patterns of specific sizes (FIG. 3b-c). We studied those patterns, as they are relevant for understanding amyloidogenesis and using STVIIE-derived amyloids in nanotechnology applications. STVIIE, when incubated in the conditions described, forms amyloid fibrils with a characteristic twist periodicity and well-defined macroscopic structure. Using JPK analysis software (JPK Instruments AG, Berlin, Germany), it is possible to determine the amyloid fibrils width, height and twist periodicity values (FIG. 3d-e). Here ΔX is established as the average distance between helical turns and ΔY as is the average distance between the maximum height and the minimum height of the helical turn. STVIIE amyloid fibrils have a periodic width of 118±16 nm, a ΔX of 170±7 nm, a ΔY of 3.2±0.4 nm and a height of 13.5±0.9 nm, within typical values.

The formation of aggregates by STVIIE quickly leads the larger elements to deposit from solution. This hinders circular dichroism studies of STVIIE secondary structure. For this reason Fourier transform infra-red (FTIR) spectroscopy was used instead (FIG. 3f-g). The changes in secondary structure content were followed for 6 hours (FIG. 3f). Then, after 6 hours, when amyloid structures are readily visible (FIG. 3a), the experiment was terminated. This time frame is also chosen given that for nanotechnology applications short incubation periods can be necessary and/or preferable. The peptides spectra are consistent with antiparallel β-sheet/aggregated strands (1675-1695 cm⁻¹) and β-sheet (1625-1640 cm⁻¹) secondary structure being present (FIG. 3f). As time progresses the content of antiparallel β-sheet and/or aggregated strands increases but their relative ratio stabilizes in less than 3 hours (FIG. 3g). Longer incubations do not change this ratio, showing that the secondary structure relative content is not modified through time. Importantly, even at high concentrations (20 μM), these fibrils are not toxic to neuronal cell cultures (FIG. 3h), in line with other studies showing that mature amyloid fibrils are relatively innocuous (contrarily to their oligomer/protofibril precursor aggregates).

Cross β-sheet secondary structure is a good indicator of amyloid presence (FIG. 3g-h), as well as the morphologies (FIG. 3a-c) and dimensions (FIG. 3g-h) observed. To further confirm this, binding to classical amyloid dyes was tested (FIG. 3i-k). Thioflavin T (ThT) fluorescence increases upon binding to mature amyloid fibrils. In the presence of amyloid fibrils the characteristic Congo Red spectrum suffers a change that results in a maximal spectral difference at 540 nm. Both ThT fluorescence and Congo Red assays are commonly used as amyloid fibrils markers. ThT fluorescence increases due to the presence of STVIIE amyloid fibrils in solution (FIG. 3i). Congo Red also binds to STVIIE amyloid fibrils in solution (FIG. 3j). Testing amyloidogenic peptide preparations that were incubated for 2 weeks (at which time point fibril morphology is clearly evident by AFM) shows that ThT fluorescence (FIG. 3k, left) and Congo Red absorbance (FIG. 3k, right) signals vary in function of the peptide concentration (even at relatively low concentrations), as expected from a peptide forming amyloid fibrils. This shows that STVIIE peptide forms a stable non-toxic amyloid fibril structure that is not affected by fibril concentration, and which occurs and is stable in physiological conditions. All this makes STVIIE suitable for nanotechnology applications, especially as a biomaterial and/or in uses where biologically relevant and bioactive materials are required and, thus, it was decided to proceed further in that direction.

With the above in mind, STVIIE was selected for functionalization studies (FIG. 4). Briefly, as mentioned above, biotin was introduced as the functional bioactive molecule, inserted into the STVIIE peptide sequence N-terminus, either via a rigid (Tdts) or a flexible (PEG9) linker and incubated as above. Since modifications to peptide sequence may hinder full amyloidogenesis, the first assay to be conducted was an investigation of fibril morphology through time, via AFM (FIG. 4a-b). With both linkers, STVIIE functionalized with biotin displayed amyloid-like morphology. However, the presence of amyloid-like morphology in AFM sample images does not preclude toxic oligomers/protofibrils from being present (either due to incomplete fibrillization or lack of fibril stability leading to its disassembly). For this reason the PEG9 and Tdts biotinylated peptide preparations were allowed to fibrilize for two weeks (in the same conditions as with non-biotinylated STVIIE peptide) and then tested regarding their toxicity (FIG. 4 c-d). Human neuroglioma H4 cells were treated with 0.2, 2.0 and 20 μM of the biotinylated forms of the peptide and the toxicity accessed by the percentage of cell death (cells viability determined by measuring LDH release). Incubations with buffer served as a baseline evaluation of naturally occurring death events and Triton was used as a positive control achieving 100% cell death. The results show that both TdTs (FIG. 4c) and (PEG9) (FIG. 4d) linkers have no major toxicity effects on cells, even at high concentrations (20 μM), as observed for free biotin peptide (FIG. 3h). In all cases cell death is at the same levels as the buffer control (10-15%), with no evolution through time (from 6 to 24 hours). This shows that biotinylated versions of STVIIE are not cytotoxic, as desired. The interaction of both candidate peptide preparations (TdTs and PEG9) with Congo Red amyloid binding dye was investigated to ascertain their amyloid properties (FIG. 4e-f). The Tdts linker peptide (FIG. 4e, dark green line) has no spectral change when compared to Congo Red dye control (FIG. 4e, black line). This indicates that the Tdts linker peptide has poor or atypical amyloid structure. As for the peptide with a PEG9 linker, this was clearly able to bind Congo Red, displaying a major spectral change, peaking at 540 nm, as expected from an amyloid fibril structure (FIG. 4e, dark red line). Moreover, the interaction of biotin-PEG9-STVIIE peptide preparation with Congo Red is directly dependent on the amount of peptide in solution (FIG. 4f, round red points), contrarily to biotin-Tdts-STVIIE (FIG. 4f, square gray points). All this supports biotin-PEG9-STVIIE amyloid fibrils as a more adequate biomaterial in the following stages.

Given the above, biotin-PEG9-STVIIE preparations were further studied (FIG. 5). Concerning ThT binding, 2-week-old preparations display a clear increase in fluorescence signal that is proportional to the amount of peptide in solution (FIG. 5a). In terms of secondary structure, evaluated via Fourier Transform Infra Red (FITR) spectroscopy (FIG. 5b), the observations echo the results obtained with (non-biotinylated) STVIIE fibrils (confront with FIG. 3f). Addition, via a PEG9 linker, of a biotin moiety to STVIIE N-terminus results in biotin functionalized STVIIE amyloid fibrils. These fibrils show both the 43-sheet and the antiparallel cross β-sheet peaks, being quite similar to the spectra obtained with free (non-biotinylated) STVIIE. Then, the activity of the bioactive molecule (biotin) attached to the fibrils was tested, namely the ability to bind its target bridge element molecule (streptavidin), when free in solution (FIG. 5c-g). Measuring the intrinsic fluorescence of the tryptophan of streptavidin shows that the biotin moiety remains functional and accessible within the amyloid fibril, binding free streptavidin and changing its fluorescence spectra (FIG. 5c). Concentrations of peptide and/or biotin range from 0 to 13.6 μM (CO to C11). Briefly, the fluorescence intensity spectra of 0.8 μM streptavidin in solution, treated with buffer (FIG. 5c, leftmost graph), free biotin (FIG. 5c, second graph from the left), non-biotinylated free amyloid peptide (FIG. 5c, third from the left) or biotinylated amyloidogenic peptide fibrils (FIG. 5c, rightmost graph) were compared. A drop on the fluorescence signal (decreased quantum yield) accompanied by peak shifting towards the left (blue shift) indicates streptavidin binding to biotin (FIG. 5c). By normalizing the fluorescence intensity to the maximum peak of the spectra (FIG. 5d), this becomes more readily visible, showing that biotinylated peptide binds to streptavidin almost as well as a positive control with only free biotin (i.e., without any peptide). Samples treated with buffer or with non-biotinylated peptide (negative controls) do not suffer peak shift or fluorescence intensity changes. It is thus clear that biotinylated peptide binds free streptavidin. To investigate this further, we calculated the differential intensity spectra (FIG. 5e) between streptavidin and free biotin curves (FIG. 5c, left) and between free (non-biotinylated) and biotinylated peptide (FIG. 5c, right). From this calculation was derived the total area (FIG. 5f) from the biotin differential spectra (blue) and biotinylated peptide differential spectra (green), showing a typical saturation binding profile. Then, the peak maximum shift was also analysed (FIG. 5g): the addition of free biotin (FIG. 5g, orange curve) or biotinylated peptide (FIG. 5g, red curve) shows that biotin causes a 10 nm blue shifted peak maximum, achieving saturation at, roughly, 4 μM. The addition of biotinylated peptide to streptavidin produces a similar blue shift of the fluorescence emission maximum (from 340 to 330 nm), as observed when free biotin is added. The shift is more gradual in the assays with biotinylated peptide, but stabilizes with the same 10 nm difference as in free biotin addition.

Overall, streptavidin fluorescence emission spectra in the presence of free biotin and of biotinylated peptide reveal similar profiles, demonstrating that the biotin moiety that is linked to the peptide fibrils binds to the streptavidin free in solution, as intended. Moreover, that binding to free streptavidin is almost as large as that of free biotin moieties.

Following, the binding of the biotinylated peptide fibril preparations to immobilized streptavidin was investigated, by observing infrared spectral changes occurring on immobilized streptavidin, as a result of interaction with biotin ligands (FIG. 5h-i). In short, by integrating the area of the peaks (FIG. 5h), we can see the kinetics of the binding of biotinylated STVIIE fibrils and the lack of binding of non-biotinylated STVIIE peptide fibrils. This is performed by immobilizing streptavidin through a covalent bond (with lysine residues) on a germanium crystal surface (that is simultaneously studied and analysed via attenuated total reflectance in a FTIR spectrometer). After zeroing the streptavidin spectra, the signal is stable when a solution of free STVIIE (FIG. 5h, black) is flushed through the system, but the signal increases with the introduction of its biotinylated form (FIG. 5h, blue). This demonstrates that only the biotinylated peptide fibrils bind to immobilized streptavidin. A control experiment, where PEG9-STVIIE amyloid fibrils are flushed through the immobilized streptavidin surface after it has been already saturated with biotin shows no interaction (FIG. 5h, red), as expected. Therefore, the recorded changes are specific for the interaction between the streptavidin on the surface and the biotin from the peptide. Close inspection of the biotinylated peptide fibrils spectrum (FIG. 5i) during its interaction with the streptavidin-labelled surface shows the occurrence of conformational changes, which further demonstrates the binding. The presence of a “shoulder peak” in the amide I region, found in amyloid fibrils, is further evidence of the biotinylated amyloid fibrils binding to the surface immobilized streptavidin molecules.

Finally, it was necessary to demonstrate that biotinylated fibrils such as the ones designed can be employed in nanotechnology, for example, in signal amplification assays. This approach was followed, first in the context of antibody-mediated recognition events of a specific ligand, in a dot-blot immunoassays test format (FIG. 6). To achieve this, amyloid fibrils were directly applied on a membrane and then a reporter conjugated molecule composed of streptavidin linked to horseradish peroxidise (S-HRP) enzyme was added, treated with substrate and imaged via chemiluminescence (FIG. 6a). This produces a luminescence signal proportional to the amount of biotin-PEG9-STVIIE fibrils on the membrane (FIG. 6b). Non-functionalized STVIIE fibrils reveal no signal. This proof-of-concept shows that the proposed approach is feasible. To further demonstrate the approach applicability in immunoassays, namely the ability to bind, detect and amplify the signal due to the presence of immobilized primary antibodies, secondary streptavidin-labelled antibodies, raised against primary antibodies IgG of different species, were employed as schematized (FIG. 6c-d). Several species of commonly used primary and secondary antibodies were used (FIG. 6e-g). A total of eight combinations of commonly used primary and secondary antibodies are detected by using the functionalized amyloid fibrils, showing the approach general applicability in immunoassays. It clearly shows the ability to target, identify and discriminate among several proteins (in this case the several immobilized IgG tested), while also giving rise to a high enough signal. The approach can thus be used with other proteins, namely of biomedical interest.

Therefore, to fully confirm with a relevant biological sample, the approach was used to detect another protein glial fibrillary acidic protein (GFAP), a glial specific protein. First, in a direct (with a single antibody) immunochemistry dot blot assay, GFAP was immobilized on a membrane surface, detected by a specific streptavidin-derivatized anti-GFAP antibody and, then, the biotin labelled amyloid fibrils are used for signal amplification (of single detection events), lowering detection thresholds (FIG. 7a). The experiment was then repeated with GFAP, in an indirect immunohistochemistry dot blot assay (with two antibodies), in a manner by which streptavidin-derivatized secondary anti-IgG antibody links to an anti-GFAP IgG antibody (FIG. 7b). In this case, a higher signal amplification is produced, further lowering detection thresholds.

The approach was then tested in a direct blot assay with biopsy-like samples, i.e., tissue extracts from mice, namely liver, where GFAP is mostly absent, and brain, where it is abundant (FIG. 7c-e). The amounts of material in the brain and liver samples are calculated based on Bradford's protein assay quantification method. The antibody was incubated with the sample, then, with the STVIIE-PEG9-biotin fibrils (which bind to the streptavidin of the antibody) and, finally, to with S-HRP (that binds the fibrils), as schematized (FIG. 7c). Although the amount of total liver and brain sample extracts is relatively low, being actually lower than the amount of peptide employed for the proof-of-concept assays (FIG. 6), the results show that, using S-HRP at 1:15000 dilution (from a 1 mg/mL stock), the signal obtained is stronger for brain samples, including with low levels of protein present (FIG. 7d-e). In short, detection of GFAP is achieved even with levels of total sample present very low, namely 10 ng (FIG. 7d) and 50 ng (FIG. 7e). There is GFAP present at much higher levels in the brain, as expected, but also a concentration-dependent signal, as desirable. This supports the approach applicability in biopsy-like tissue samples.

At this point, with all the data and information described from FIG. 1 to FIG. 7, the usefulness of amyloid species as biomaterials, the approach general applicability and the gains of using amyloid fibrils in the detection and signal amplification of biomarkers are all demonstrated. To go further, variations on this common theme were performed, first by producing biotin-PEG13-STVIIE amyloid fibrils. Biotin-PEG13-STVIIE is incubated and allowed to from amyloid fibrils, as shown by (a) AFM (FIG. 8a), that also bind streptavidin free in solution (FIG. 8b-d), in a concentration-dependent manner (FIG. 8e). This shows that, when free in solution, the biotin moiety of biotin-PEG13-STVIIE amyloid fibrils remains active and functional. Then, the activity of immobilized biotin-PEG13-STVIIE amyloid fibrils was tested (FIG. 8f-h). Following experimental setting optimization (FIG. 8f-g), it is shown that, 60 minutes of incubation with 1 μg/mL of functionalized enzyme (S-HRP) allows even 20 ng of immobilized biotin-PEG13-STVIIE immobilized amyloid fibrils to be detected (FIG. 8h). The interaction is specific since pure fibrils of free STVIIE peptide (i.e., not derivatized with biotin) give no signal (FIG. 8h). Then, as a final test, it is shown that biotin-PEG13-STVIIE fibrils can detect/amplify the presence of immobilized proteins (FIG. 8i). Essentially, even 2 ng (faint signal) of streptavin-derivatized anti-GFAP antibody are detected (FIG. 8i). Importantly, there is specificity, as BSA protein (negative control, not derivatized) shows no signal (FIG. 8i). This validates the approach with a different form of the biomaterials.

Another variation concerns using mixed preparations of amyloid fibrils (FIG. 9), containing biotin-derivatized amyloid peptides as well as the corresponding free peptide version (i.e., without biotin). Briefly, biotin-PEG9-STVIIE peptide monomers are incubated with free STVIIE peptide monomers for 2 weeks at different ratios and evaluated by AFM (FIG. 9a). Amyloid-like fibrils are seen after 2 weeks incubation and these readily bind Congo Red (FIG. 9b), displaying also a typical FTIR spectra (FIG. 9c-d), as the peaks are consistent with cross beta-sheet structure (FIG. 9d). When these 2-week old fibrils are immobilized on membranes, preparations containing at time zero 70% free STVIIE and 30% biotin-PEG9-STVIIE (mass percentages of peptide monomers), give an excellent signal after being treated with S-HRP and tested in a dot blot assay (FIG. 9e). Moreover, immobilizing preparations of biotin-PEG13-STVIIE peptide monomers incubated with free STVIIE peptide monomers shows that, after one-week co-incubation, such mixed peptides preparations are also easily detectable via dot blot assay (FIG. 9f). This is especially true for the 70% free STVIIE and 30% biotin-PEG13-STVIIE ratio (mass percentages of peptide monomers). The mixed preparation with more free peptide gives an excellent signal after S-HRP treatment, and with only one-week incubation (in other immunoassays, fibrils were at least 2 weeks old). Possibly, the free STVIIE peptide gives a structure core, more hydrophobic, with the biotinylated peptide being on the fridges of the fibril, which can result in more biotin moieties available for binding.

Salmonella spp. is also detected (namely Salmonella enterica Tiphymurium), via amyloid based signal amplification, in an indirect ELISA test format, as shown in the experiment schematics (FIG. 10a), and corresponding results (FIG. 10b). The experimental results shown correspond to 11 conditions tested (varying primary antibody concentration, fibril amounts and, also, enzyme quantities). The best condition tested was C6. This corresponds to 1 μg/mL of streptavidin-labeled primary antibody, pre-incubated (RT, 60′) with 2 μg/mL of biotinylated peptide amyloid fibrils (biotin-PEG13-STVIEE, allowed to fibrilize for at least 2 weeks). This preparation was then allowed to interact with samples (60′, 37° C.), after which 1 μg/mL of S-HRP was added (60′, 37° C.), before measuring absorbance at 450 nm. A concentration as low as 10^{\hat{ }6} Salmonella spp. colony forming units per mL was detected in about 5 hours, total experiment time, an improvement from current immunology based assays, which, to achieve near that, require, previous to the actual immunoassay, 8 to 48 hours pre-incubation and enrichment of the sample with the target bacteria. Thus, results require about 2 working days, while in our setting they are obtained within the same day, after less than 6 hours, a clear improvement.

The results of using other amyloidogenic sequences besides STVIEE are also shown here, all incubated in physiological conditions as described above (FIGS. 11, 12 and 13). Thus, these may also lead to similar findings. In addition, as an example, a schematic of the incorporation into a multi-sensing biosensor is shown, in a different setup (FIG. 14a). This can be used for sensing multiple molecules in the same platform test assay (FIG. 14b), with sample analytes, fibrils/protofibrils, and, finally detection reagents entering from a collection chamber through the opening indicated by the upper arrow and leaving via the lower arrow. The collection chamber schematic design is fully explained in detail, namely the location of key valves (FIG. 15a) as well as the flow direction (FIG. 15b). To clarify: the interaction and detection cell chamber mentioned in FIG. 15 corresponds to FIG. 14; the L symbol in FIG. 14a represents a generic ligand, while multiple different ligands are represented in FIG. 14b by L1, L2, etc. up to L7. Thus, the events described in FIG. 14a are contained and contained within the immediate region of the symbol L1, L2, etc., up to L7, of FIG. 14b, marked in blue. Similarly, FIG. 14b multiplex is, as mentioned, within FIG. 15 (interaction and detection cell chamber). Finally, another example of a detection system is shown (FIG. 16). In this setting, after a ligand is immobilized (i), a primary (ii) and then a secondary antibody (iii) are connected to the ligand. The streptavidin-labelled secondary antibody binds biotinylated amyloid fibrils (iv), which through their multiple biotin moieties, are able to simultaneously bind to strepavidinated magnetic nanoparticles (v), allowing magnetoresistive force discrimination to be performed. All these are alternatives to the ones already mentioned, showing the potential of this amyloid based technology.

Amyloid toxicity, although initially thought to be caused by mature fibrils, has been demonstrated to be mostly associated with the fibrils precursors (oligomers and protofibrils)¹⁻¹⁰. This knowledge, alongside with amyloid fibrils chemical and mechanical stability, triggered the interest in amyloid fibrils as biomaterials. For such purposes, short amyloid peptides are better than longer expensive sequences. It is also important that amyloid fibrils that are formed and remain stable in physiological pH and temperature conditions. Moreover, they should be modifiable with chemical moieties to add them new functions if desired.

The data provided herein demonstrate that, among other candidate peptides, STVIIE is able to form stable fibrils in physiological conditions. The data also demonstrate that biotin can be added to the N-terminus of the STVIIE peptide via a PEG9 linker, resulting in mature and well-structured amyloid fibrils. It is also shown that not only does this modified peptide remain able to form amyloid fibrils but also that it acquires a new function, becoming able to bind to free and immobilized streptavidin.

By employing streptavidin labelled antibodies and reporter molecules, the data demonstrate that the biotinylated peptide produced can be employed to detect the glial fibrillar acidic protein at low concentrations, inclusively in cell extracts. Moreover, the inventors have also demonstrated the ability of the biotinylated peptide to bind and detect other immobilized proteins, namely biologically relevant IgG molecules. The data yet further show that biotin-PEG13-STVIIE, as well as mixed preparations, of biotin derivatized and of free peptide, form amyloid fibrils that detect and enable signal amplification in immunoassays.

A bacteria, Salmonella spp., was also detected, namely in an ELISA format, via the amyloid-based nanomaterials. These comprehensive data show the biological activity, usefulness, and applicability of the described biomaterial, enabling nanotechnology applications employing these and similar peptides (modified and functionalized, as described or in similar ways), namely for uses in biosensing and/or signal amplification technologies.

It is understood that the Examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

### REFERENCES

1. Rousseau F, Schymkowitz J, Serrano L. Protein aggregation and amyloidosis: confusion of the kinds? Curr Opin Struct Biol. 2006; 16(1):118-126. doi:10.1016/j.sbi.2006.01.011
2. Walsh D M, Hartley D M, Kusumoto Y, et al. Amyloid beta-protein fibrillogenesis. Structure and biological activity of protofibrillar intermediates. J Biol Chem. 1999; 274(36):25945-25952. http://www.ncbi.nlm.nih.gov/pubmed/10464339.
3. Petkova A T, Ishii Y, Balbach J J, et al. A structural model for Alzheimer's beta-amyloid fibrils based on experimental constraints from solid state NMR. Proc Natl Acad Sci USA. 2002; 99(26):16742-16747. doi:10.1073/pnas.262663499
4. Carrio M M, Villaverde A. Protein aggregation as bacterial inclusion bodies is reversible. FEBS Lett. 2001; 489(1):29-33. http://www.ncbi.nlm.nih.gov/pubmed/11231008.
5. Bousset L, Thomson N H, Radford S E, Melki R. The yeast prion Ure2p retains its native alpha-helical conformation upon assembly into protein fibrils in vitro. EMBO J. 2002; 21(12):2903-2911. doi:10.1093/emboycdf303
6. Padrick S B, Miranker A D. Islet amyloid: phase partitioning and secondary nucleation are central to the mechanism of fibrillogenesis. Biochemistry. 2002; 41(14):4694-4703. http://www.ncbi.nlm.nih.gov/pubmed/11926832.
7. Dobson C M. Protein misfolding, evolution and disease. Trends Biochem Sci. 1999; 24(9):329-332. http://www.ncbi.nlm.nih.gov/pubmed/10470028.
8. Chiti F, Dobson C M. Protein Misfolding, Amyloid Formation, and Human Disease: A Summary of Progress Over the Last Decade. Annu Rev Biochem. 2017; 86(1):27-68. doi:10.1146/annurev-biochem-061516-045115
9. Bellotti V, Chiti F. Amyloidogenesis in its biological environment: challenging a fundamental issue in protein misfolding diseases. Curr Opin Struct Biol. 2008; 18(6):771-779. doi:10.1016/j.sbi.2008.10.001
10. Sarkar N, Dubey V K. Exploring critical determinants of protein amyloidogenesis: a review. J Pept Sci. 2013; 19(9):529-536. doi:10.1002/psc.2539
11. Martins I C, Kuperstein I, Wilkinson H, et al. Lipids revert inert Abeta amyloid fibrils to neurotoxic protofibrils that affect learning in mice. EMBO J. 2008; 27(1):224-233. doi:10.1038/sj.emboj.7601953
12. Luheshi L M, Dobson C M. Bridging the gap: from protein misfolding to protein misfolding diseases. FEBS Lett. 2009; 583(16):2581-2586. doi:10.1016/j.febslet.2009.06.030
13. Makin O S, Atkins E, Sikorski P, Johansson J, Serpell L C. Molecular basis for amyloid fibril formation and stability. Proc Natl Acad Sci USA. 2005; 102(2):315-320. doi:10.1073/pnas.0406847102
14. Gebbink M F, Claessen D, Bouma B, Dijkhuizen L, Wosten H A. Amyloids—a functional coat for microorganisms. Nat Rev Microbiol. 2005; 3(4):333-341. doi:10.1038/nrmicro1127
15. Talbot N J. Aerial morphogenesis: enter the chaplins. Curr Biol. 2003; 13(18):R696-8. http://www.ncbi.nlm.nih.gov/pubmed/13678605.
16. Romero D, Aguilar C, Losick R, Kolter R. Amyloid fibers provide structural integrity to Bacillus subtilis biofilms. Proc Natl Acad Sci USA. 2010; 107(5):2230-2234. doi:10.1073/pnas.0910560107
17. Iconomidou V A, Vriend G, Hamodrakas S J. Amyloids protect the silkmoth oocyte and embryo. FEBS Lett. 2000; 479(3):141-145. http://www.ncbi.nlm.nih.gov/pubmed/10981723.
18. Podrabsky J E, Carpenter J F, Hand S C. Survival of water stress in annual fish embryos: dehydration avoidance and egg envelope amyloid fibers. Am J Physiol Regul Integr Comp Physiol. 2001; 280(1):R123-31. http://www.ncbi.nlm.nih.gov/pubmed/11124142.
19. Cherny I, Gazit E. Amyloids: not only pathological agents but also ordered nanomaterials. Angew Chem Int Ed Engl. 2008; 47(22):4062-4069. doi:10.1002/anie.200703133
20. Gras S L, Squires A M, Dobson C M, MacPhee CE. Functionalised fibrils for bio-nanotechnology. 2006 Int Conf Nanosci Nanotechnology, Vols 1 2. 2006:265-267.
21. Jung J P, Gasiorowski J Z, Collier J H. Fibrillar peptide gels in biotechnology and biomedicine. Biopolymers. 2010; 94(1):49-59. doi:10.1002/bip.21326
22. Knowles T P, Buehler M J. Nanomechanics of functional and pathological amyloid materials. Nat Nanotechnol. 2011; 6(8):469-479. doi:10.1038/nnano.2011.102
23. Knowles T P, Vendruscolo M, Dobson C M. The amyloid state and its association with protein misfolding diseases. Nat Rev Mol Cell Biol. 2014; 15(6):384-396. doi:10.1038/nrm3810
24. Maji S K, Perrin M H, Sawaya M R, et al. Functional amyloids as natural storage of peptide hormones in pituitary secretory granules. Science (80-). 2009; 325(5938):328-332. doi:10.1126/science.1173155
25. Badtke M P, Hammer N D, Chapman M R. Functional amyloids signal their arrival. Sci Signal. 2009; 2(80):pe43. doi:10.1126/scisignal.280pe43
26. Berson J F, Harper D C, Tenza D, Raposo G, Marks M S. Pme117 initiates premelanosome morphogenesis within multivesicular bodies. Mol Biol Cell. 2001; 12(11):3451-3464. http://www.ncbi.nlm.nih.gov/pubmed/11694580.
27. Rochin L, Hurbain I, Serneels L, et al. BACE2 processes PMEL to form the melanosome amyloid matrix in pigment cells. Proc Natl Acad Sci USA. 2013; 110(26):10658-10663. doi:10.1073/pnas.1220748110
28. Hauser C A E, Maurer-Stroh S, Martins I C. Amyloid-based nanosensors and nanodevices. Chem Soc Rev. 2014; 43(15):5326-5345. doi:10.1039/c4c500082j
29. Morris K, Serpell L. From natural to designer self-assembling biopolymers, the structural characterisation of fibrous proteins & peptides using fibre diffraction. Chem Soc Rev. 2010; 39(9):3445-3453. doi:10.1039/b919453n
30. Hauser C A, Zhang S. Designer self-assembling peptide nanofiber biological materials. Chem Soc Rev. 2010; 39(8):2780-2790. doi:10.1039/b921448 h
31. Loo Y, Zhang S, Hauser C A. From short peptides to nanofibers to macromolecular assemblies in biomedicine. Biotechnol Adv. 2012; 30(3):593-603. doi:10.1016/j.biotechadv.2011.10.004
32. Makin O S, Serpell L C. Structures for amyloid fibrils. FEBS J. 2005; 272(23):5950-5961. doi:10.1111/j.1742-4658.2005.05025.x
33. Stromer T, Serpell L C. Structure and morphology of the Alzheimer's amyloid fibril. Microsc Res Tech. 2005; 67(3-4):210-217. doi:10.1002/jemt.20190
34. Sikorski P, Atkins E D, Serpell L C. Structure and texture of fibrous crystals formed by Alzheimer's abeta(11-25) peptide fragment. Structure. 2003; 11(8):915-926. http://www.ncbi.nlm.nih.gov/pubmed/12906823.
35. Bowerman C J, Nilsson B L. Self-assembly of amphipathic beta-sheet peptides: insights and applications. Biopolymers. 2012; 98(3):169-184. doi:10.1002/bip.22058
36. Maurer-Stroh S, Debulpaep M, Kuemmerer N, et al. Exploring the sequence determinants of amyloid structure using position-specific scoring matrices. Nat Methods. 2010; 7(3):237-242. doi:10.1038/nmeth.1432
37. Adamcik J, Mezzenga R. Adjustable twisting periodic pitch of amyloid fibrils. Soft Matter. 2011; 7(11):5437-5443. doi:Doi 10.1039/C1sm05382e
38. Lakshmanan A, Zhang S, Hauser C A. Short self-assembling peptides as building blocks for modern nanodevices. Trends Biotechnol. 2012; 30(3):155-165. doi:10.1016/j.tibtech.2011.11.001
39. Men D, Guo Y C, Zhang Z P, et al. Seeding-induced self-assembling protein nanowires dramatically increase the sensitivity of immunoassays. Nano Lett. 2009; 9(6):2246-2250. doi:10.1021/n19003464
40. Ayres N. Polymer brushes: Applications in biomaterials and nanotechnology. Polym Chem. 2010; 1(6):769-777. doi: Doi 10.1039/B9py00246d
41. Paul T J, Hoffmann Z, Wang C, et al. Structural and Mechanical Properties of Amyloid Beta Fibrils: A Combined Experimental and Theoretical Approach. J Phys Chem Lett. 2016; 7(14):2758-2764. doi:10.1021/acs.jpclett.6b01066
42. Ruggeri F S, Šneideris T, Vendruscolo M, Knowles T P J. Atomic force microscopy for single molecule characterisation of protein aggregation. Arch Biochem Biophys. 2019; 664:134-148. doi:10.1016/j.abb.2019.02.001
43. Nagarkar R P, Miller S E, Zhong S, Pochan D J, Schneider J P. Dynamic protein folding at the surface of stimuli-responsive peptide fibrils. Protein Sci. 2018; 27(7):1243-1251. doi:10.1002/pro.3394
44. Ouberai M M, Dos Santos A L G, Kinna S, et al. Controlling the bioactivity of a peptide hormone in vivo by reversible self-assembly. Nat Commun. 2017; 8(1). doi:10.1038/s41467-017-01114-1
45. Chan K H, Xue B, Robinson R C, Hauser C A E. Systematic Moiety Variations of Ultrashort Peptides Produce Profound Effects on Self-Assembly, Nanostructure Formation, Hydrogelation, and Phase Transition. Sci Rep. 2017; 7(1). doi:10.1038/s41598-017-12694-9
46. Dai B, Li D, Xi W, et al. Tunable assembly of amyloid-forming peptides into nanosheets as a retrovirus carrier. Proc Natl Acad Sci USA. 2015; 112(10):2996-3001. doi:10.1073/pnas.1416690112
47. Men D, Zhang Z P, Guo Y C, et al. An auto-biotinylated bifunctional protein nanowire for ultra-sensitive molecular biosensing. Biosens Bioelectron. 2010; 26(4):1137-1141. doi:10.1016/j.bios.2010.07.103
48. Poviloniene S, Časaite V, Bukauskas V, Šetkus A, Staniulis J, Meškys R. Functionalization of α-synuclein fibrils. Beilstein J Nanotechnol. 2015; 6(1):124-133. doi:10.3762/bjnano.6.12
49. Lopez de la Paz M, Serrano L. Sequence determinants of amyloid fibril formation. Proc Natl Acad Sci USA. 2004; 101(1):87-92. doi:10.1073/pnas.2634884100
50. Pastor M T, Kummerer N, Schubert V, et al. Amyloid toxicity is independent of polypeptide sequence, length and chirality. J Mol Biol. 2008; 375(3):695-707. doi:S0022-2836(07)01082-0 [pii]10.1016/j.jmb.2007.08.012
51. Rousseau F, Schymkowitz J, Da Rocha Martins 1, De Strooper B. Means and Methods for the Production of Amyloid Oligomers. 2008; (WO/2008/028939). http://www.freepatentsonline.com/WO2008028939.html.
52. Diamandis E P, Christopoulos T K. The Biotin (Strept)Avidin System—Principles and Applications in Biotechnology. Clin Chem. 1991; 37(5):625-636.
53. Faustino A F, Guerra G M, Huber R G, et al. Understanding Dengue Virus Capsid Protein Disordered N-Terminus and pep14-23-Based Inhibition. ACS Chem Biol. 2014: in press. doi:10.1021/CB500640t
54. Kuperstein 1, Broersen K, Benilova 1, et al. Neurotoxicity of Alzheimer's disease Abeta peptides is induced by small changes in the Abeta42 to Abeta40 ratio. EMBO J. 2010; 29(19):3408-3420. doi:10.1038/emboj.2010.211
55. Klunk W E, Pettegrew J W, Abraham D J. Quantitative evaluation of congo red binding to amyloid-like proteins with a beta-pleated sheet conformation. J Histochem Cytochem. 1989; 37(8):1273-1281. http://www.ncbi.nlm.nih.gov/pubmed/2666510.
56. Lai Z, Colon W, Kelly J W. The acid-mediated denaturation pathway of transthyretin yields a conformational intermediate that can self-assemble into amyloid. Biochemistry. 1996; 35(20):6470-6482. doi:10.1021/bi952501g
57. Carvalho F A, Carneiro F A, Martins I C, et al. Dengue virus capsid protein binding to hepatic lipid droplets (LD) is potassium ion dependent and is mediated by LD surface proteins. J Virol. 2012; 86(4):2096-2108. doi:10.1128/JVI.06796-11
58. Carvalho F A, Connell S, Miltenberger-Miltenyi G, et al. Atomic force microscopy-based molecular recognition of a fibrinogen receptor on human erythrocytes. ACS Nano. 2010; 4(8):4609-4620. doi:10.1021/nn1009648
59. Domingues M M, Silva P M, Franquelim H G, Carvalho F A, Castanho M A, Santos N C. Antimicrobial protein rBPI21-induced surface changes on Gram-negative and Gram-positive bacteria. Nanomedicine. 2014; 10(3):543-551. doi:10.1016/j.nano.2013.11.002
60. Diaz-Caballero et al (2018) Prion 12 5-6 266-272
61. Men et al (2010) Biosens Bioelectron 26(4) 1137-1141
62. Chan et al (2011) Anal Chem 83 9370-9377
63. CN105717287
64. Sasso et al (2014) Nanoscale 6 (3) 1629-1634
65. Guerra et al (2013) Eur Biophys 42 Suppl 1 S141

1. A functionalised biomaterial comprising aggregated self-assembling peptides, wherein at least a proportion of the self-assembling peptides are functionalised with a biological agent or a chemical agent.

2. A functionalised biomaterial according to claim 1, wherein the biomaterial is a fibril or gel.

3. A functionalised biomaterial according to claim 1, wherein the self-assembling peptides are able to self-assemble under physiological conditions.

4. A functionalised biomaterial according to claim 1, wherein the self-assembling peptides comprise a peptide that comprises or consists of STVIIE, QVQIIE, ISFLIF and/or GNNQQNY.

5. (canceled)

6. A functionalised biomaterial according to claim 1, wherein the functionalising agent is biotin.

7. A functionalised biomaterial according to claim 1, comprising at least one bridge element connected to the functionalised biomaterial, optionally by binding to the functionalising agent.

8. A functionalised biomaterial according claim 7, comprising a reporter molecule bound to at least one bridge element.

9. A functionalised biomaterial according claim 7, comprising multiple reporter molecules bound to multiple bridge elements.

10. A functionalised biomaterial according to claim 7, wherein the bridge element comprises streptavidin.

11. A functionalised biomaterial according to claim 7, comprising a recognition element that specifically binds to the peptide, the bridge element or the functionalising molecule.

12. A functionalised biomaterial according to claim 11 wherein the recognition element further comprises an analyte-binding region.

13. A functionalised biomaterial according to claim 12 wherein the analyte-binding region is antibody.

14. A functionalised biomaterial according to claim 7, wherein the functionalising molecule comprises biotin, the bridge element comprises streptavidin, the reporter molecule comprises an enzyme optionally HRP or comprises an optical label, and the recognition element comprises a streptavidin-labelled antibody.

15. A functionalised biomaterial according to claim 7, wherein the bridge element or the recognition element is in solution.

16. A functionalised biomaterial according to claim 7, wherein the bridge element or the recognition element is immobilised to a surface

17. (canceled)

18. An assay to detect an analyte, comprising contacting the analyte with: (i) a biomaterial according to claim 1; or (ii) separate components of a biomaterial according to claim 1, wherein the biomaterial is then allowed to form in situ; or (iii) amyloidogenic peptides comprising, consisting or consisting essentially of STVIIE, QVQIIE, ISFLIF or GNNQQNY, wherein the biomaterial is then allowed to form in situ.

19-21. (canceled)

22. An amyloidogenic peptide comprising, consisting or consisting essentially of STVIIE, QVQIIE, ISFLIF or GNNQQNY.

23. (canceled)

24. A method of preparing a biomaterial, comprising providing amyloidogenic peptides according to claim 22 in conditions suitable for them to self-assemble, and allowing the peptides to self-assemble to form the biomaterial, optionally wherein at least a proportion of the amyloidogenic peptides are functionalised.

25. (canceled)

26. A kit comprising one or more amyloidogenic peptides according to claim 22 and instructions for their self-assembly into a biomaterial.

27. (canceled)

28. A biosensor comprising a functionalised biomaterial according to claim 1.
